(19) United States
(12) Patent Application Publication
(10) Pub. No.: US 2017/0224613 A1
(43) Pub. Date: Aug. 10, 2017

(54) HYDROGEL COMPOSITIONS, METHODS FOR PREPARING THE SAME AND USES THEREOF

(71) Applicant: CHONGQING MJ MEDICAL DEVICES CO., LTD., Chongqing, P.R.C. (CN)

(72) Inventors: Jun WU, Chongqing (CN); Gaoxing LUO, Chongqing (CN); Johnson Yiu-Nam LAU, Newport Beach, CA (US); Manson FOK, Hong Kong (CN); Chun-Ho WONG, Hong Kong (CN); Meinrad FLURY, Kerzers (CH); Benjamin Tak-Kwong LEE, Hong Kong (CN)

(21) Appl. No.: 15/515,409

(22) PCT Filed: Sep. 30, 2014
(86) PCT No.: PCT/CN2014/088001

§ 371 Date: Mar. 29, 2017

(51) Int. Cl. A61K 9/00; A61K 9/06; A61K 47/36; A61K 31/736; A61K 31/738; A61K 9/51; A61K 31/045

(52) U.S. Cl. CPC A61K 9/0014; A61K 9/5161; A61K 9/06; A61K 31/045; A61K 31/736; A61K 31/738; A61K 47/36

(57) ABSTRACT

Disclosed are hydrogel composition, method for preparing the same and use thereof. The hydrogel composition comprises a guar gum derivative which is crosslinked, a mint-based component, water, and optionally, an amphiphilic core-shell nanoparticle having a hydrophobic core and a hydrophilic shell, wherein the amount of the mint-based component is in the range of 1-6 wt % and the amount of the water is in the range of 65-95 wt %, based on the total weight of the hydrogel composition. The hydrogel composition is safe and can provide symptomatic relief for patients with symptoms such as severe and intractable pruritus associated with hypertrophic scars. The symptomatic relief effect of the hydrogel composition is reproducible, and is independent of the etiology of the burn trauma, extent of the scarring and duration of the scar formation, while tachyphylaxis is not observed. Also disclosed is a medical device comprising the hydrogel composition.

### FIELD OF THE INVENTION

This invention relates to pharmaceutical compositions and uses thereof. In particular, the invention relates to hydrogel compositions, methods for preparing the hydrogel compositions, hydrogel compositions for use in relieving symptoms induced by hypertrophic scars, as well as methods and medical device for relieving such symptoms.

### BACKGROUND OF THE INVENTION

Burn-related injury is a major global health issue that is associated with an estimated 265,000 deaths annually reported by the World Health Organization (WHO Fact Sheet 2014). In particular, the majority of the deaths occurred in low and middle-income countries. In China, there are around 15-20 million burn cases every year (incidence rate varies from 0.5-1.5%, depending on the criteria) and an estimated 2-3 million burn patients require hospitalization.

When normal skin tissue is damaged in burn, injury, disease or infection, the body repairs itself with the formation of scars. In some patients with genetic predisposition, or in cases of deep thickness skin loss that can be caused by severe burns, hypertrophic scars may be resulted. The natural wound repair processes are often associated with pain and pruritus. In patients with hypertrophic scars related to severe burns, it is always pruritus, rather than pain, that continues to be a major clinical issue. One of the major consequences of severe pruritus is the disturbance of sleep, which in severe cases can cause sleep deprivation, poor quality of life, and depression. As of today, no consistently effective treatment for the symptomatic relief of pruritus in these patients known to the inventors has been found. Various treatment modalities have been proposed anecdotally and most were not tested in controlled clinical studies^{[1-6]}.

Chinese patent publication No. CN103340843A discloses a composite latex comprising 40-52 wt % of crosslinked guar gum, 30-42 wt % of poly(methyl acrylate)-chitosan nanoparticles and 6-18 wt % of methyl salicylate as main components, as well as certain amounts of heparin, allantoin, menthol, camphor and borneol as additional components. However, the water content is not specified, and it is speculated that no workable gel can be obtained according to such disclosure, because generally the nanoparticles at the high level of 30-42 wt % cannot be obtained practically. Also, the composite latex does not contain any glycerol, and it is speculated that the latex will not form a gel with acceptable texture. Published reports also suggested that hydrogel sheeting is at least as effective as silicone gel in the management of hypertrophic scars^{[7]} with only a few showed that silicone gel sheets could reduce pruritus^{[8, 9]}.

However, no effective treatment modality known to the inventors is currently available for symptomatic control of pruritus for most patients. The existing medical products for patients with hypertrophic scars cannot provide desired symptomatic relief and long-term effect, and may cause side effects such as irritation.

### SUMMARY OF THE INVENTION

### Technical Problems to be Solved

One object of the present invention is to provide a hydrogel composition effective in symptomatic relief for patients with hypertrophic scars. Another object of the present invention is to provide a method for preparing the hydrogel composition. Still another object of the present invention is to provide a hydrogel composition for use in relieving symptoms induced by hypertrophic scars. Still another object of the present invention is to provide a method and a medical device for relieving symptoms induced by hypertrophic scar.

### Technical Solutions

Recognizing the sufferings of severe symptoms such as pruritus with sleep disturbance in the patients with hypertrophic scars and in order to achieve the above mentioned objects, the present inventors have performed thorough theoretical and experimental investigations. The inventors have, surprisingly, found that oil-based and air-tight bandages or gels, such as silicone gel, should be avoided, and that steroid creams and anti-histamine-based creams are generally ineffective for patients with hypertrophic scars. The inventors have further found that water-based formulation is better than oil-based formulation in the symptomatic relief of hypertrophic scars, which was not found to be reported in the prior art. Based on the investigations and findings, the present invention is completed.

Accordingly, the present invention provides a hydrogel composition comprising:

a guar gum derivative which is crosslinked;

a mint-based component;

water; and

optionally, an amphiphilic core-shell nanoparticle having a hydrophobic core and a hydrophilic shell;

wherein the amount of the mint-based component is in the range of 1-6 wt % and the amount of the water is in the range of 65-95 wt %, based on the total weight of the hydrogel composition.

Preferably, the amount of the guar gum derivative is in the range of 1-7 wt % based on the total weight of the hydrogel composition.

Preferably, the guar gum derivative is selected from the group consisting of hydroxypropyl guar gum, carboxymethyl guar gum, O-carboxymethyl-O-hydroxypropyl guar gum and O-2-hydroxy-3-(trimethylammonio)propyl guar.

Preferably, the guar gum derivative is a combination of hydroxypropyl guar gum and carboxymethyl guar gum, and the amount of the hydroxypropyl guar gum is in the range of 1-6 wt % and the amount of the carboxymethyl guar gum is in the range of more than 0 wt % to 1 wt %, based on the total weight of the hydrogel composition.

Preferably, the guar gum derivative is crosslinked by borate, glutaraldehyde and/or glyoxal.

Preferably, the guar gum derivative is at least partially further crosslinked with the shell of the nanoparticle.

Preferably, the amount of the nanoparticle is in the range of 0.5-2 wt % based on the total weight of the hydrogel composition.

Preferably, the nanoparticle is selected from the group consisting of poly(methyl methacrylate)-chitosan nanoparticle, poly(methyl methacrylate)-polyethylenimine nanoparticle and poly(methyl methacrylate)-poly(allylamine) nanoparticle.

Preferably, the nanoparticle is poly(methyl methacrylate)-chitosan nanoparticle, and wherein the poly(methyl methacrylate) constitutes the core of the nanoparticle and the chitosan constitutes the shell of the nanoparticle.

Preferably, the weight ratio of the poly(methyl methacrylate) to the chitosan is in the range of 1: (3-5).

Preferably, the number average particle size of the nanoparticle is in the range of 100-150 nm.

Preferably, the mint-based component is encapsulated by the nanoparticle.

Preferably, the mint-based component is selected from the group consisting of peppermint oil, menthol, cornmint oil and spearmint oil.

Preferably, the mint-based component is a combination of peppermint oil and additional menthol, and wherein the amount of the peppermint oil is in the range of more than 0 wt % to 4 wt % and the amount of the additional menthol is in the range of 0.5-2 wt %, based on the total weight of the hydrogel composition.

Preferably, the hydrogel composition further comprises up to 2.5% methyl salicylate based on the total weight of the hydrogel composition.

Preferably, the hydrogel composition comprises 1-2.5 wt % of methyl salicylate based on the total weight of the hydrogel composition.

Preferably, the hydrogel composition does not comprise methyl salicylate.

Preferably, the hydrogel composition further comprises a plasticizer.

It is another aspect of the present invention to provide a method for preparing the hydrogel composition according to the invention, comprising the steps of:

1) providing an emulsion containing at least a part of the mint-based component; and

2) combining the emulsion containing at least a part of the mint-based component with the guar gum derivative, the remaining mint-based component if any, and a crosslinker to induce crosslinking of the guar gum derivative, thereby forming the hydrogel composition.

Preferably, the step 1) comprises the following steps:

i) providing an aqueous emulsion of the amphiphilic core-shell nanoparticle having a hydrophobic core and a hydrophilic shell; and

ii) mixing the aqueous emulsion of the nanoparticle with at least a part of the mint-based component to form the emulsion containing at least a part of the mint-based component.

Preferably, the concentration of the aqueous emulsion of the nanoparticle provided in step i) is in the range of 0.5-5% (w/w).

Preferably, in step ii) the aqueous emulsion of the nanoparticle and the at least a part of the mint-based component are mixed such that the mint-based component is encapsulated by the nanoparticle.

Preferably, the amount of the mint-based component added in step 1) is in the range of 20-100 wt % based on the total amount of the mint-based component.

Preferably, the amount of the mint-based component added in step 1) is in the range of 20-30 wt % based on the total amount of the mint-based component.

Preferably, the mint-based component added in step 1) is menthol, and the remaining mint-based component added in step 2) is peppermint oil.

Preferably, the step 1) further comprises adding methyl salicylate.

Preferably, the step 2) further comprises adding a plasticizer.

It is yet another aspect of the present invention to provide the hydrogel composition according to the invention for use in relieving symptoms induced by hypertrophic scars.

Preferably, the dosage of the hydrogel composition is in the range of 0.2-0.8 g/cm² skin.

Preferably, the hydrogel composition is in the form of dressings.

It is yet another aspect of the present invention to provide a method of relieving symptoms induced by hypertrophic scars, comprising: administrating to a subject in need of relieving the symptoms a pharmaceutically effective amount of the hydrogel composition according to the invention.

Preferably, the hydrogel composition is administered at a dosage in the range of 0.2-0.8 g/cm² skin.

Preferably, the hydrogel composition is topically administered at a dosage in the range of 0.2-0.8 g/cm² skin and renewed every 4-6 hours.

It is yet another aspect of the present invention to provide a medical device comprising:

a package containing the hydrogel composition according to the invention, and

an instruction for administration of the hydrogel composition for relieving the symptoms induced by hypertrophic scars.

Preferably, the package further comprises a substrate to which the hydrogel composition is disposed, or an applicator for application of the hydrogel composition.

### Advantageous Effects

The present invention provides a hydrogel composition which is safe and can provide symptomatic relief for patients with symptoms such as severe and intractable pruritus associated with hypertrophic scars. The symptomatic relief effect of the hydrogel composition is reproducible, and is independent of the etiology of the burn trauma, extent of the scarring and duration of the scar formation, while tachyphylaxis is not observed. The active ingredients in the hydrogel composition are synergistic even if they are present at low levels. Since the hydrogel composition comprises little or no methyl salicylate, irritation can be suppressed or even obviated. On the other hand, since the hydrogel composition comprises a large amount of water, it is more flexible and can provide better cooling effect and excellent conformity to various contours of hypertrophic scars. The organic components of the hydrogel composition can be encapsulated in amphiphilic core-shell nanoparticles and the hydrogel composition is an aqueous composition, so that the unpleasant oily feeling is minimized and the symptomatic relief effect can last for relatively long time, and delivery of active ingredients is controlled. The hydrogel composition of the present invention can be used as an occlusive patch that can adapt to any contour of hypertrophic scars.

### BRIEF DESCRIPTION OF THE DRAWINGS

Preferred embodiments of the present invention will be explained by way of examples and with reference to the accompanying drawings, in which:

FIG. 1 comprises diagrams showing results of evaluation on warming sensation, cooling sensation and skin irritation respectively in the healthy volunteer study.

FIG. 2 is a diagram showing changes in the JW scale with CQ-01 (A) versus the gel alone without additives (B; gel control) and with gauze cover alone (C; negative control) (per protocol analysis; n=70; error bar=SD).

FIG. 3 is a diagram showing the effect of repeated application of CQ-01 to the same patients (n=10 for the first course, n=10 for the second course, and n=3 for the third course). It is noted that there is a trend that the second application (n=10) had a stronger effect from 6 hours to Day 3. It is also noted that there is a trend that the third application had a stronger effect from 12 hours to Day 7 (n=3).

FIG. 4 is a diagram showing the percentage of patients with a drop of 20, 30, and 40 points in JW score after CQ-01 application.

FIG. 5 is a flow chart showing a proposed treatment paradigm for patients with severe pruritus associated with burn-induced hypertrophic scars.

FIG. 6 is a diagram showing the efficacy of gel control vs. silicone gel sheet vs. gauze only control in a follow-up study.

### DETAILED DESCRIPTION OF THE PREFERRED EMBODIMENTS

In order to make those skilled in the art have a better understanding of the technical solutions of the present invention, more detailed description is provided below with reference to specific embodiments and the accompanying drawings. It is to be understood that the present invention is not limited to the particular compositions, methodologies, embodiments or protocols described herein.

As used in this specification and the appended claims, the singular forms “a”, “an”, and “the” include plural referents unless the content clearly dictates otherwise. Thus, for example, reference to a composition containing “a compound” includes a mixture of two or more compounds, and reference to a particle or nanoparticle includes a plurality of particles or nanoparticles. As used in this specification and the appended claims, the term “or” is generally employed in its sense including “and/or” unless the content clearly indicates otherwise.

As used in this specification and the appended claims, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

All numbers herein are assumed to be modified by the term “about”. The recitation of numerical ranges by endpoints includes all numbers subsumed within that range (e.g., 1 to 5 includes 1, 1.5, 2, 2.75, 3, 3.80, 4, and 5). Percentages of material amounts are by weight unless otherwise indicated.

The present description refers to a number of chemical terms and abbreviations used by those skilled in the art. Nevertheless, definitions of selected terms are provided for clarity and consistency.

The term “hydrogel” or “gel” as used herein refers to a material forming, to various degrees, a jelly-like product when suspended in an aqueous medium, typically water. Typically, a hydrogel may comprise a polymeric material which can include a crosslinked macromolecular network, which exhibits the ability to swell in water and to retain a significant portion of water within its structure without dissolving.

The term “crosslinked” or “crosslinking” as used herein refers to any means effective to cause a material to form a hydrogel in an aqueous medium (typically water), and may refer to the case of being partially crosslinked and the case of being totally crosslinked. Such means can include, for example, physical entanglement, crystalline domains, covalent bonds, ionic complexes and associations, hydrophilic associations such as hydrogen bonding, and hydrophobic associations or Van der WaaIs forces, Typically, the means involves the use of a crosslinker capable of crosslinking two or more molecules (e.g., polymeric chains) such that covalent bonds are formed among the molecules.

The term “amphiphilic” as used herein refers to a material having both hydrophilic and hydrophobic moieties.

The term “hydrophobic”, “hydrophobic moiety” or “hydrophobic group” refers to those compounds, groups or moieties being immiscible in water.

The term “hydrophilic”, “hydrophilic moiety” or “hydrophilic group” refers to those compounds, groups or moieties being miscible in water.

The term “encapsulated” (and any form of encapsulated, such as “encapsulate” or “encapsulating”) as used herein refers to molecular entrapment of small organic compounds.

The term “scar” as used herein refers to a wound or a wound surface that is closed by regrowth of an epithelial barrier.

The term “hypertrophic scar” as used herein includes a scar characterized by thick, raised scar tissue that stays essentially within the boundaries of the original injury, Hypertrophic scars generally contain characteristic nodules, and may result from a full-thickness injury, such as a burn accident.

The term “derivative” as used herein includes any compound that is made from a base compound, for example, by replacing one atom in the base compound with another atom or group of atoms.

The term “nanoparticle” as used herein includes those having a number average particle size (Dₙ) ranging from 1 nm to less than 1000 nm, for example, 200 nm, 150 nm, 100 nm, etc. The particle size can be measured by a particle size analyzer, for example, Coulter LS230.

The term “borate” or “boric acid-based compound” as used herein includes those capable of providing borate anions or groups for crosslinking of a guar gum derivative. For example, the borate may be sodium borate, borax (Na₂B₄O₇.10H₂O), potassium borate, boric acid, alkyl borates such as trimethyl borate, phenyl borates, or combinations thereof.

As used herein, the term “mint-based component” or “mint-based components” refers to compound or compounds made synthetically or obtained (e.g., extracted) from cornmint, peppermint or other mints.

As used herein, the terms “effective amount” and “pharmaceutically effective amount” refer to the amount of an active agent required to be administered in order to induce a desired result in the patient. That result may be alleviation or relief (complete or partial) of the symptoms or condition of irritation, pain, pruritus, and/or tingling of a scar.

As used herein, the term “plasticizer” refers to a chemical that increases the rheological property of the hydrogel.

As used herein, the term “degree of substitution” with regard to a guar gum derivative refers to the average number of substituents attached per monosaccharides unit. For example, degree of substitution of hydroxypropyl group with regard to hydroxypropyl guar gum refers to the average number of hydroxypropyl group on each monosaccharides unit.

In the present invention, the degree of substitution of a guar gum derivative is in the range of 0 to 3, preferably in the range of 0.4 to 2.

The present invention provides a hydrogel composition comprising: a guar gum derivative which is crosslinked, a mint-based component, and water; wherein the amount of the mint-based component is in the range of 1-6 wt % and the amount of the water is in the range of 65-95 wt %, based on the total weight of the hydrogel composition.

Since the hydrogel composition comprises a comparatively large amount of water, it is more flexible to be applied to various contours of scars and can provide better cooling effect and excellent conformity. However, excessively high amount of water will result in a very watery product and not suitable for application. Preferably, the amount of the water is in the range of 70-80 wt %, and most preferably in the range of from about 75 wt % to 77 wt %, based on the total weight of the hydrogel composition.

It is found that the guar gum-based hydrogel has an excellent safety profile and the resulting occlusive gel patch can provide maximum contact exposure for the hypertrophic scars. In addition, the guar gum-based hydrogel can retain a large amount of water, which is desired in this invention. It is also found that the hydrogel is non-irritating and non-sticky to the skin surface and can be readily applied and removed. In the hydrogel composition according to the present invention, the amount of the guar gum derivative is preferably in the range of 1-7 wt %, more preferably in the range of 4-6 wt %, and most preferably in the range of from about 5.5 wt % to 5.8 wt %, based on the total weight of the hydrogel composition.

Guar gum refers to mucilage found in the seed of the leguminous plant Cyamopsis tetragonolobus, The water soluble fraction (85%) thereof is called “guaran”, which consists of linear chains of (1,4)-β-D-rnannopyranosyl units-with α-D-galactopyranosyl units attached by (1,6) linkages. The ratio of D-galactose to D-mannose in guaran is about 1:2. Guar gum typically has a weight average molecular weight of between 2,000,000 and 5,000,000 Daltons.

Guar seeds are composed of a pair of tough, non-brittle endosperm sections, hereafter referred to as “guar splits”, between which is sandwiched the brittle embryo (germ). After dehulling, the seeds are split, the germ (43-47% of the seed) is removed by screening. The splits typically contain about 78-82% galactomannan polysaccharide and minor amounts of some proteinaceous material, inorganic salts, water-insoluble gum, and cell membranes, as well as some residual seedcoat and seed embryo.

Processes for making derivatives of guar gum splits are generally known in the art, for example, as described in CN101490093A, which is incorporated herein by reference, Typically, guar splits are reacted with one or more derivatizing agents under appropriate reaction conditions to produce a guar polysaccharide having the desired substituent groups. Suitable derivatizing reagents are commercially available and typically contain a reactive functional group, such as an epoxy group, a chlorohydrin group, or an ethylenically unsaturated group, and at least one other substituent group, such as a cationic, nonionic or anionic substituent group, or a precursor of such a substituent group per molecule, wherein the substituent group may be linked to the reactive functional group of the derivatizing agent by bivalent linking group, such as an alkylene or oxyalkylene group. Suitable cationic substituent groups include primary, secondary, or tertiary amino groups or quaternary ammonium, sulfonium, or phosphinium groups. Suitable nonionic substituent groups include hydroxyalkyl groups, such as hydroxypropyl groups. Suitable anionic groups include carboxyalkyl groups, such as carboxymethyl groups. The cationic, nonionic and/or anionic substituent groups may be introduced to the guar polysaccharide chains via a series of reactions or by simultaneous reactions with the respective appropriate derivatizing agents.

In one embodiment, the guar splits react with an alkylene oxide derivatizing agent, such as ethylene oxide, propylene oxide, or butylene oxide, under known alkoxylation conditions to add hydroxyalkyl and/or poly(alkyleneoxy) substituent groups to the guar polysaccharide chains.

In one embodiment, the guar splits react with a carboxylic acid derivatizing agent, such as sodium monochloroacetate, under known esterification conditions to add carboxyalkyl groups to the guar polysaccharide chains.

In one embodiment, the derivatizing agent comprises a cationic substituent group that comprises a cationic nitrogen radical, more typically, a quaternary ammonium radical. Typical quaternary ammonium radicals are trialkylammonium radicals, such as trimethylammonium radicals, triethylammonium radicals, tributylammonium radicals, aryldialkylammonium radicals, such as benzyldimethylammonium radicals and ammonium radicals in which the nitrogen atom is a member of a ring structure, such as pyridinium radicals and imidazoline radicals, each in combination with a counterion, typically a chloride, bromide, or iodide counterion. In one embodiment, the cationic substituent group is linked to the reactive functional group of the cationizing agent by an alkylene or oxyalkylene linking group.

It is preferably that the guar gum derivative is selected from hydroxypropyl guar gum (HPG), carboxymethyl guar gum (CMG), O-carboxymethyl-O-hydroxypropyl guar gum (CMHPG), O-2-hydroxy-3-(trimethylammonio)propyl guar (HTPG), etc. Among them, the combination of hydroxypropyl guar gum (HPG) and carboxymethyl guar gum (CMG) is most preferred because HPG is safe, low cost, and common while CMG has ionic interaction with the chitosan surface of nanoparticle as described below. In such a preferred embodiment, the amount of the hydroxypropyl guar gum can be in the range of 1-6 wt %, more preferably in the range of 4-6 wt %, and most preferably in the range of from about 4.9 wt % to 5.2 wt %; the amount of the carboxymethyl guar gum can be in the range of more than 0 wt % to 1 wt %, more preferably in the range of 0.4-0.8 wt %, and most preferably in the range of from about 0.5 wt % to 0.6 wt %, based on the total weight of the hydrogel composition.

The guar gum derivative can be crosslinked by borate, for example, as described in US patent application No. 20130330430A1, which is incorporated herein by reference. For example, hydroxypropyl guar gums react with borates through hydroxyl groups such that they are crosslinked. Other useful crosslinkers include, for example, glutaraldehyde, glyoxal, etc. In the present invention, the boric acid-based compounds are preferred to be used as a crosslinker.

Preferably, the hydrogel composition of the present invention may further comprise an amphiphilic core-shell nanoparticle having a hydrophobic core and a hydrophilic shell.

More preferably, the guar gum derivative in the hydrogel composition is at least partially further crosslinked with the shell of the amphiphilic core-shell nanoparticle, such that a more stable hydrogel can be provided as, for example, carboxymethyl group in CMG can form ionic crosslink with ammonium group in chitosan in the nanoparticle.

In the hydrogel composition of the present invention, the amount of the amphiphilic core-shell nanoparticle is preferably in the range of 0.5-2.0 wt %, more preferably in the range of 1.0-1.5 wt %, most preferably 1.25 wt %, based on the total weight of the hydrogel composition.

The amphiphilic core-shell nanoparticle may be prepared by those processes known in the art, for example, as described in U.S. Pat. No. 6,573,313B2, which is incorporated herein by reference. The amphiphilic core-shell nanoparticle may be prepared by a process comprising treating a water-soluble polymer containing amino groups with a small amount of alkyl hydroperoxide in the presence of a vinylic monomer, such that nanoparticles comprising a hydrophobic polymer as the core and a hydrophilic polymer as the shell are produced.

The water-soluble polymer forming the shell portion of the nanoparticles may be selected from natural and synthetic water-soluble polymers containing amino groups, including, for example, polyethyleneimine, and other synthetic amino polymers; chitosan and other N-acetyl sugars; and casein, gelatin, bovine serum albumin and other proteins.

The monomer forming the core portion of the nanoparticles may be selected from hydrophobic vinylic monomer, acrylate monomer, acrylamide monomer, polymerizable nitrile, acetate, and chloride monomers, a styrenic monomer, and a diene monomer.

Examples of vinylic monomers include those of formula R¹R²C═CH₂, where R¹ is hydrogen or alkyl, and where R² is alkyl, aryl, heteroaryl, halo, cyano, or other suitable hydrophobic group. Preferred groups for R¹ include hydrogen and methyl. Preferred groups for R² include C₁-C₆ alkyl; phenyl; monocyclic heteroaryl with 4 to 8 ring atoms, more preferably 5 or 6 ring atoms, and with 1, 2 or 3 ring heteroatoms, preferably 1 or 2, more preferably 1 ring atom, being selected from nitrogen, oxygen or sulfur; chloro; and cyano.

Examples of dienes include those represented by the following formula: CH₂═C(R¹)—C(R²)═CH₂, where R¹ is hydrogen or halogen or alkyl, and where R² is hydrogen or alkyl, especially C₁-C₆ alkyl. Preferred groups for R¹ include hydrogen, chloride and methyl. Preferred groups for R² include hydrogen and methyl.

Examples of acrylate monomers include those of formula CH₂═CR³COOR⁴, where R³ is hydrogen or alkyl, and where R⁴ is alkyl or substituted allyl, or other suitable hydrophobic group. Preferred groups for R³ include hydrogen and methyl. Preferred groups for R⁴ include C₁-C₁₆, more preferably C₁-C₁₂, alkyl which may be straight-chain or branched, and such groups substituted with one or more substituents chosen from unsubstituted amino, monosubstituted amino or disubstituted amino, hydroxy, carboxy, or other usual acrylate substituent. Particular acrylate monomers include methyl methacrylate, ethyl (meth)acrylate, isopropyl (meth)acrylate, n-butyl (meth)acrylate, and the like.

Examples of acrylamide monomers include those of formula CH₂═CR³COONHR⁴, where R³ and R⁴ are as defined above.

The polymerized vinylic monomer may have a polydispersity, M_{w}/Mₙ, in the range of from 1.5 to 3. The weight % of the monomer forming the core portion or the hydrophobic polymer may range from 25% to 95% of the total weight of the core-shell nanoparticles. The water-soluble polymer containing amino groups may be present in an amount of from 5 to 75% by weight, based on the total weight of the core-shell nanoparticles.

The amphiphilic core-shell nanoparticle is preferably selected from poly(methyl methacrylate)-chitosan nanoparticle (PMMA-chitosan nanoparticle), poly(methyl methacrylate)-polyethylenimine nanoparticle (PMMA-PEI nanoparticle), poly(methyl methacrylate)-poly(allylamine) nanoparticle (PMMA-PAA nanoparticle), etc. Among them, the PMMA-chitosan nanoparticle is most preferred, wherein the PMMA constitutes the core of the nanoparticle and the chitosan constitutes the shell of the nanoparticle. The chitosan shell may, for example, at least partially crosslink with the carboxymethyl group of carboxymethyl guar gum, thereby providing a more stable hydrogel. The weight ratio of the PMMA to the chitosan is preferably in the range of 1: (3-5), most preferably 1:4.

The number average particle size (Dₙ) of the amphiphilic core-shell nanoparticles is preferably in the range of 100-150 nm.

The amphiphilic core-shell nanoparticles can serve as vehicles for controlled delivery of oily, hydrophobic compounds. The mint-based components in the hydrogel composition are preferably encapsulated in the hydrophobic core of the nanoparticles. In this event, the miscibility of organic compounds in the hydrogel composition is enhanced and unpleasant oily feeling is minimized to optimize patients' comfort and the symptomatic relief effect can last for relatively longer time. The delivery of active ingredients can also be controlled. In addition, the hydrophilic shell such as chitosan shell of the nanoparticles enables the nanoparticles to be embedded or dispersed in the aqueous environment of the hydrogel composition without phase-separation.

In the hydrogel composition of the present invention, the amount of the mint-based component is in the range of 1-6 wt % based on the total weight of the hydrogel composition. Excessive mint-based component will results in irritation. Preferably, the mint-based component includes peppermint oil, menthol, cornmint oil, spearmint oil, etc. Among them, the combination of peppermint oil and additional menthol is most preferred. In such a preferred embodiment, the amount of the peppermint oil is preferably in the range of more than 0 wt % to 4 wt %, more preferably 2-4 wt %, most preferably 3.6 wt %, based on the total weight of the hydrogel composition; the amount of the additional menthol is preferably in the range of 0.5-2 wt %, more preferably 1-1.5 wt %, most preferably 1.4 wt %, based on the total weight of the hydrogel composition.

As well known in the art, peppermint oil is an essential oil comprising menthol (for example, about 50 wt %). Thus, as used herein, the term “peppermint oil” refers to an essential oil obtained (e.g., extracted) from peppermint which comprises a certain amount of menthol. The term “menthol” used in parallel with peppermint oil refers to a separated, substantially pure compound, (1R,2S,5R)-2-isopropyl-5-methylcyclohexanol, or a mixture of stereoisomers thereof, which is used in addition to the peppermint oil. As used herein, the term “amount of the additional menthol” refers to the amount of the menthol used in addition to the peppermint oil.

Surprisingly, the amounts of peppermint oil and menthol according to the present invention synergistically provide cooling sense and anti-pruritic effect so as to achieve extreme symptomatic relief for patients with hypertrophic scars even though they are present at low levels and there is no other active ingredient in the hydrogel composition. However, it will be appreciated by a person skilled in the art that the hydrogel composition of the present invention may also comprise any other known ingredients for relieving symptoms induced by hypertrophic scars, such as heparin, allantoin, camphor, borneol, or combinations thereof.

The hydrogel composition according to the present invention may comprise up to 2.5 wt %, for example 1-2.5 wt %, methyl salicylate based on the total weight of the hydrogel composition. It is found that if the amount of the methyl salicylate is above 2.5 wt %, the composition would be irritating to skin, particularly to burn patients. In one embodiment, the hydrogel composition does not comprise methyl salicylate.

In a preferred embodiment of the present invention, the hydrogel composition further comprises a plasticizer. The plasticizer may facilitate the hydrogel composition according to the present invention to form a gel with a desirable and patient-friendly texture and appearance. For example, useful plasticizers include, but not limited to, glycerol, diglycerol, poly(ethylene glycol), propylene glycol, Methylene glycol, mannitol, sorbitol, xylitol, urea, oligomeric lactic acid and citric acid. The amounts of the plasticizer can be determined depending on practical applications. For example, based on the total weight of the hydrogel composition, the amount of plasticizer may range from 1-10 wt %, such as 4-6 wt %.

The hydrogel composition may further comprise one or more selected from emulsifiers, preservatives, rheology modifiers and buffers. The examples of useful emulsifiers, preservatives, rheology modifiers and buffers are known to the person skilled in the art.

Emulsifiers may be optionally used to enhance the stability of the hydrogel composition. For example, useful emulsifiers include, but not limited to, Cremophor EL, Cremophor RH, phospholipids, propylene glycol, polysorbate, poloxamer, polyethylene glycol and its derivatives, lecithin, tween, span, and glyceryl monostearate.

Preservatives may be optionally used to inhibit the growth of microorganisms in the hydrogel composition. Useful preservatives include, but not limited to, methyl paraben, propyl paraben, C₁₂ to C₁₅ alkyl benzoates, alkyl p-hydroxybenzoates, aloe vera extract, ascorbic acid, benzalkonium chloride, benzoic acid, benzoic acid esters of C₉ to C₁₅ alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, phenoxyethanol, potassium sorbate, silicone oils, sodium propionate, sodium benzoate, sodium bisulfite, sorbic acid, stearic fatty acid, vitamin E, vitamin E acetate and derivatives, esters, salts and mixtures thereof.

Rheology modifiers may be optionally used to modify viscosity and flow of the hydrogel composition. Useful rheology modifiers include, but not limited to, zinc sulfate heptahydrate, zinc oxide, glycerol, carbomers, acrylic copolymers, polyacrylamides, polysaccharides, natural gums, caprylic/capric triglyceride (Migliol 810), isopropyl myristate (IM or IPM), ethyl oleate, triethyl citrate, dimethyl phthalate, benzyl benzoate, and the like.

Buffers may be optionally used to control pH of the hydrogel composition. Preferably, the buffers buffer the composition from a pH of about 5-8, more preferably from a pH of about 6-7, and most preferably from a pH of about 6.0-6.5, Useful buffers include, but not limited to, phosphate buffer, citrate buffer, acetate buffer, cacodylic acid buffer, 2-(N-morpholino)ethanesulfonic acid (MES) buffer, carbonate buffer, Bis(2-hydroxyethyl)-amino-tris(hydroxymethyl)-methane (Bis-tris) buffer, N-(2-acetamido)iminodiacetic acid (ADA) buffer, Bis-tris propane buffer, piperazine-N,N′-bis(2-ethanesulfonic acid) (PIPES) buffer, imidazole buffer, N,N-bis (2-hydroxyethyl) 2-aminoethane sulphonic acid (BES) buffer, 4-Morpholinepropanesulfonic acid (MOPS) buffer, N-[Tris(hydroxymethyl)methyl]-2-aminoethanesulfonic acid (TES) buffer, 4-(2-hydroxyethyl)-1-piperazineethanesulfonic acid (HEPES) buffer, N-(2-Hydroxyethyl)piperazine-N-2-hydroxypropanesulfonic acid (HEPPSO) buffer, triethanolamine buffer, tartarate buffer, tricine buffer, tromethamine (TRIS) buffer, glycine amide buffer, bicine buffer, glycylglycine buffer, and N-Tris(hydroxymethyl)methyl-3-aminopropanesulfonic acid (TAPS) buffer.

The amounts of the emulsifier, preservative, rheology modifier and buffer can be determined depending on practical applications. For example, based on the total weight of the hydrogel composition, the amount of emulsifier may range from 0-5 wt %, such as 2 wt %; the amount of preservative may range from 0-1 wt %, such as 0.33 wt %; the amount of rheology modifier may range from 0-7 wt %, preferably 1-7 wt %, such as 5.8 wt %; the amount of buffer may range from 0-3 wt %, such as 1.5 wt %.

In one preferred embodiment, the hydrogel composition according to the present invention comprises, based on the total weight of the hydrogel composition, 1.4 wt % menthol, 3.6 wt % peppermint oil, 2.5 wt % methyl salicylate, 1.25 wt % PMMA-chitosan nanoparticle, 5.2 wt % HPG, 0.55 wt % CMG and 74.22 wt % water.

In another preferred embodiment, the hydrogel composition according to the present invention comprises, based on the total weight of the hydrogel composition, 1.4 wt % menthol, 3.6 wt % peppermint oil, 0 wt % methyl salicylate, 1.25 wt % PMMA-chitosan nanoparticle, 5.2 wt % HPG, 0.55 wt % CMG and 76.72 wt % water.

In still another preferred embodiment, the hydrogel composition according to the present invention comprises, based on the total weight of the hydrogel composition, 1.4 wt % menthol, 3.6 wt % peppermint oil, 1.5 wt % methyl salicylate, 1.25 wt % PMMA-chitosan nanoparticle, 5.2 wt % HPG, 0.5 wt % CMG and 76.97 wt % water.

Another aspect of the invention provides a method for preparing the hydrogel composition according to the present invention, comprising:

1) providing an emulsion containing at least a part of the mint-based component; and

2) combining the emulsion containing at least a part of the mint-based component with the guar gum derivative, the remaining mint-based component if any, and a crosslinker to induce crosslinking of the guar gum derivative, thereby forming the hydrogel composition.

Preferably, the step 1) comprises:

i) providing an aqueous emulsion of the amphiphilic core-shell nanoparticle having a hydrophobic core and a hydrophilic shell; and

ii) mixing the aqueous emulsion of the nanoparticle with at least a part of the mint-based component to form the emulsion containing at least a part of the mint-based component.

Preferably, the concentration of the aqueous emulsion of the nanoparticle in step i) is in the range of 0.5-5% (w/w), preferably 3-5% (w/w), more preferably 4% (w/w).

Preferably, in step ii) the aqueous emulsion of the nanoparticle and the at least a part of mint-based component are mixed such that the mint-based component is encapsulated by the nanoparticle.

The mint-based component of the hydrogel composition may be added entirely in step 1). Alternatively, the mint-based component may be divided into two portions, one being added in step 1) and the remaining being added in step 2). Preferably, the amount of the mint-based component added in step 1) is in the range of 20-100 wt %, more preferably 20-30 wt %, based on the total amount of the mint-based component. More preferably, the mint-based component added in step 1) is menthol and the remaining mint-based component added in step 2) is peppermint oil.

In one embodiment, the method may further comprise adding methyl salicylate in step 1), more preferably in step ii). As described above, the hydrogel composition according to the present invention may comprise up to 2.5 wt %, for example 1-2.5 wt %, methyl salicylate based on the total weight of the hydrogel composition. However, the amount of the methyl salicylate cannot exceed 2.5 wt %, for which would result in irritating to skin, particularly to burn patients. Thus, it is preferred that no methyl salicylate is added.

In a preferred embodiment, the method may further comprise adding a plasticizer in step 2). For example, useful plasticizers include, but not limited to, glycerol, diglycerol, poly(ethylene glycol), propylene glycol, Methylene glycol, mannitol, sorbitol, xylitol, urea, oligomeric lactic acid and citric acid. The amount of the plasticizer added may range from, for example, 1-10 wt %, such as 4-6 wt % based on the total weight of the hydrogel composition.

The method may further comprise adding one or more selected from emulsifiers, preservatives, rheology modifiers and buffers. The species and the amounts of these optional components are as described above.

In the method according to the invention, all the steps may be carried out at room temperature unless clearly dictated otherwise.

Another aspect of the invention provides the hydrogel composition according to the present invention for use in relieving symptoms induced by hypertrophic scars. Said symptoms may include, for example, pruritus, pain, etc.

The hydrogel composition may be used in a form of, for example, dressing, such as gel sheet or any other useful forms. The size of the sheet may be, but not limited to, 3-7 mm in thickness, 80-120 mm in width and 100-200 mm in length. Other size dimensions may be used as well according to practical applications. The administration dosage of the hydrogel composition is an effective amount for relieving the symptoms. For example, the dosage of the hydrogel composition may be in the range of 0.2-0.8 g/cm² skin, preferably 0.4-0.6 g/cm² skin.

Another aspect of the invention provides a method of relieving symptoms induced by hypertrophic scars, comprising the step of administrating to a subject in need of relieving the symptoms a pharmaceutically effective amount of the hydrogel composition according to the present invention.

As described above, the dosage of the hydrogel composition may be, for example, in the range of 0.2-0.8 g/cm² skin, preferably 0.4-0.6 g/cm² skin.

In a particular embodiment, the hydrogel composition is topically administered at a dosage in the range of 0.2-0.8 g/cm² skin and renewed every 4-6 hours.

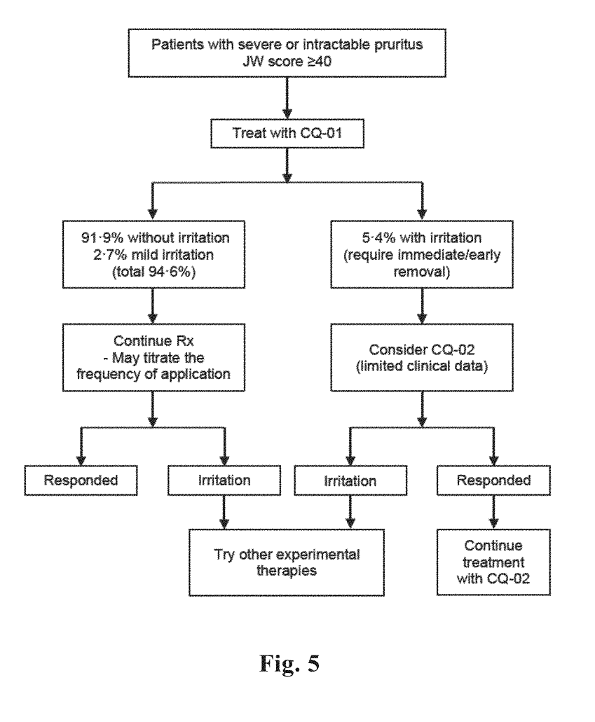

In a more particular embodiment, particularly for a patient with hypertrophic scars who is sensitive to any stimulation, the hydrogel composition may be administered according to the algorithm as shown in FIG. 5.

Another aspect of the invention provides a medical device comprising:

a package containing the hydrogel composition according to the present invention, and

an instruction for administration of the hydrogel composition for relieving the symptoms induced by hypertrophic scars.

The hydrogel composition according to the present invention may be formulated on the spot or pre-formulated as desired. The package containing the hydrogel composition may further comprise a substrate to which the hydrogel composition is disposed, for example, the substrate may be a gauze that is coated or impregnated with the hydrogel composition. Alternatively, the package containing the hydrogel composition may further comprise an applicator capable of facilitating the application of the hydrogel composition onto the skin of a subject.

Advantages and embodiments of the present invention are further illustrated by the following examples with reference to the figures, but the particular materials and amounts thereof recited in these examples, as well as other conditions and details, should not be construed to unduly limit the invention.

### EXAMPLES

The materials and devices used in the examples are described below:

L-menthol: available from Essencia

verdox: available from Essencia

methyl salicylate: available from Spectrum chemicals and laboratory products

Cremophor EL: available from BASF

hydroxypropyl guar: available from Polygal

carboxymethyl guar: available from Polygal

methyl paraben: available from Goldward Chemicals Ltd

propyl paraben: available from Goldward Chemicals Ltd

borax: available from Sigma-aldrich

boric acid: available from Sigma-aldrich

monosodium phosphate: available from Sigma-aldrich

zinc sulphate heptahydrate: available from Spectrum chemicals and laboratory products

zinc oxide: available from Sigma-aldrich

peppermint oil: available from Essencia

glycerol: available from Goldward Chemicals Ltd

ultrasonicator: available from Syclon; Type SKLON-3200D

homogenizer: available from ShangHai Angni Instruments & meters Co., Ltd; Type AE300S-P

### Example 1: Preparation of Hydrogel Composition

Preparation of 4% (w/w) PMMA-Chitosan Nanoparticles Emulsion:

The raw PMMA-chitosan nanoparticles emulsion was prepared according to Prof. Li's method described in U.S. Pat. No. 6,573,313B2, wherein the number average particle diameter of the nanoparticles used in this example is 144 nm. Purified water was added to dilute the raw nanoparticles to 4% (w/w).

Emulsion Preparation:

1.4 g L-menthol and 0.1 g verdox were mixed with 1.5 g methyl salicylate using an ultrasonicator in 40 kHz for 30 min. 2.0 g Cremophor EL was added and the obtained mixture was stirred mildly to give a pale yellow solution. Then 31.25 g of the 4% (w/w) nanoparticles emulsion was added to the pale yellow solution and stirred violently with a homogenizer for 5 minutes to give a white emulsion A,

Powder Mix 1 Preparation:

5.2 g hydroxypropyl guar (Degree of substitution of hydroxypropyl group=0.4, viscosity of 2% (w/w) solution after 2 hour is 8520 mPa·s, using Brookfield BV viscometer, 20 rpm, Spindle 2 at 25° C.), 0.55 g carboxymethyl guar (Degree of substitution of carboxymethyl group=0.2, viscosity of 3% (w/w) solution after 2 hour is 30650 mPa·s, using Brookfield BV viscometer, 20 rpm, Spindle 2 at 25° C.), 0.22 g methyl paraben, 0.11 g propyl paraben and 0.75 g borax were mixed to give a pale yellow fine powder.

Powder Mix 2 Preparation:

0.8 g boric acid, 1.5 g monosodium phosphate, 0.05 g zinc sulphate heptahydrate and 0.15 g zinc oxide were mixed to give a white fine powder.

Preparation of Hydrogel Composition:

40 g purified water was added to the white emulsion A obtained above and stirred mildly for 2 minutes to give a white emulsion B. Powder mix 1 was added to this emulsion B and stirred violently for 5 minutes to give a paste-like mixture. Then powder mix 2 was added to the paste-like mixture and stirred violently for 5 minutes to give a small-sized hydrogel. Finally, 3.6 g peppermint oil, 5.6 g glycerol and 5.22 g water were added and stirred violently for 10 minutes to obtain the target hydrogel (pH=6.2).

### Examples 2-7 and Comparative Examples 1-2

The hydrogel composition of Examples 2-7 and Comparative Examples 1-2 were prepared in the same manner as described in Example 1 except that the contents of indicated components were changed as shown in Table 1 below, the remainder being other additives including preservative, buffer, rheology modifier and plasticizer.

TABLE 1
Contents (wt %) of different components of the compositions of Examples 1-7 and Comparative Examples 1-2

| | nanoparticle | L- | peppermint | methyl | hydroxypropyl | carboxymethyl | | Cremophor | Crosslinker: | Crosslinker: |
|---|---|---|---|---|---|---|---|---|---|---|
| | wt %/Dₙ (nm) | menthol | oil | salicylate | guar | guar | water | EL | Boric acid | Borax |
| Example 1 | 1.25/144 | 1.4 | 3.6 | 1.5 | 5.2 | 0.55 | 75.22 | 2.0 | 0.80 | 0.75 |
| Example 2 | 1.25/144 | 1.4 | 3.6 | 2.5 | 5.2 | 0.55 | 74.22 | 2.0 | 0.80 | 0.75 |
| (CQ-01) | | | | | | | | | | |
| Example 3 | 1.25/144 | 1.4 | 3.6 | 0 | 5.2 | 0.55 | 76.72 | 2.0 | 0.80 | 0.75 |
| (CQ-02) | | | | | | | | | | |
| Example 4 | 0.5/144 | 0.5 | 0.5 | 0 | 1.0 | 0 | 95.00 | 0.5 | 0 | 0.50 |
| Example 5 | 2.0/144 | 2.0 | 4.0 | 1.5 | 6.0 | 1.0 | 65.00 | 3.0 | 0.80 | 1.00 |
| Example 6 | 1.25/144 | 1.4 | 0 | 1.0 | 5.2 | 0.55 | 70.15 | 2.0 | 0.80 | 0.75 |
| Example 7 | 0 | 1.4 | 3.6 | 0 | 4.0 | 0 | 82.00 | 1.0 | 0 | 0.50 |
| Comparative | 1.25/144 | 1.4 | 0 | 11 | 4.9 | 0.55 | 70 | 2 | 1.3 | 0 |
| Example 1 | | | | | | | | | | |
| Comparative | 1.25/144 | 1.4 | 0 | 11 | 5.2 | 0.55 | 69.70 | 2 | 1.3 | 0 |
| Example 2 | | | | | | | | | | |

### Experimental Example 1: WVTR Tests of Hydrogel Compositions

The hydrogel composition prepared in Example 2 (called CQ-01) was produced into a sheet with a thickness of 5 mm and the water vapor transmission rate (WVTR) was evaluated as an indication of the breathability of the CQ-01 sheet using a modified ASTM E-96 method at 45-55% relative humidity and 37° C. The test environment is realized in a testing chamber. Its temperature is remained within ±: 1° C. and humidity within ±: 5%. The hydrogel is of even thickness, and avoiding edge leaking is of great significance. The edges of the specimen are sealed using parafilm to prevent the passage of vapor into, or out of, or around the specimen edges.

A silicone gel (available from Nanyang Huibo Biotechnology Co., Ltd.: Wei Shi Road, Industrial Park, Nanyang, Henan Province, China) sheet with a thickness of 1.5 mm was also evaluated on WVTR, and both closed system (with double plate sealers) and open system were used as negative and positive controls respectively.

It was found that CQ-01 sheet with a thickness of 5 mm had an ideal water vapor transmission rate of 1730±323 g·m⁻²·24 h⁻¹, with closed system control being 49±11 g·m⁻²·24 h⁻¹ and open system being 9861±188 g·m⁻²·24 h⁻¹, A silicone gel sheet of 1.5 mm thick had a WVTR of only 144±21 g·m⁻²·24 h⁻¹. The CQ-01 hydrogel also had a better breathability than regular skin based on water vapor transmission rate (204 g·m⁻²·24 h⁻¹)³⁸. This result indicated that CQ-01 is a good dressing material for closed wound application.

The WVTR tests were also performed on the hydrogel compositions prepared in other Examples in the same manner. It has been found that these hydrogel compositions all exhibited acceptable water vapor transmission properties (VVVTR: greater than 500 g·m⁻²·24 h⁻¹) and are good materials for closed wound application.

### Experimental Example 2: Clinical Studies

1. Testing in Healthy Volunteers

In a pilot study to evaluate the hydrogel composition formulations, the different hydrogel prototypes (with a gauze on which the respective hydrogel composition was disposed) of Examples 2 (called gel B in Table 3) and 6 (called gel C in Table 3) and Comparative Example 1 (called gel A in Table 3) were tested on 8 healthy volunteers (M:F=5:3, ages 27-64), respectively. It is well established that the hypertrophic scars of patients can be very sensitive to touch or chemical irritations/medications, Therefore, the objective of this testing was to select the prototype with the maximum cooling effect and a minimal amount of irritation. Three clinical parameters were considered including the cooling sensation, the warming sensation and irritation/erythema on the applied skin area. In this pilot study, each participant has applied the topical hydrogels (120 mm×100 mm×5 mm; 70 g) on to their forearm (one gel per forearm at a time) and scored their subjective warming and cooling sensations at 5, 10, 20, and 30 min after hydrogel application according to Table 2. The skin irritation reaction was also examined and scored by a researcher according to Table 2.

TABLE 2
Subjective sensation scoring system for warming sensation,
cooling sensation and grading of skin irritation reaction

| Warming sensation | Cooling sensation | Skin irritation |
|---|---|---|
| 0 = neutral | 0 = neutral | 0 = normal skin |
| 1 = slightly warm | 1 = slightly cool | 1 = mild erythema |
| 2 = warm | 2 = cool | 2 = mild erythema with mild |
| | | pain |
| 3 = hot | 3 = cold | 3 = mild erythema with |
| | | moderate pain |
| 4 = very hot | 4 = very cold | 4 = moderate erythema with |
| | | moderate pain |
| | | 5 = severe erythema with severe |
| | | pain |
| | | 6 = severe erythema with blister |

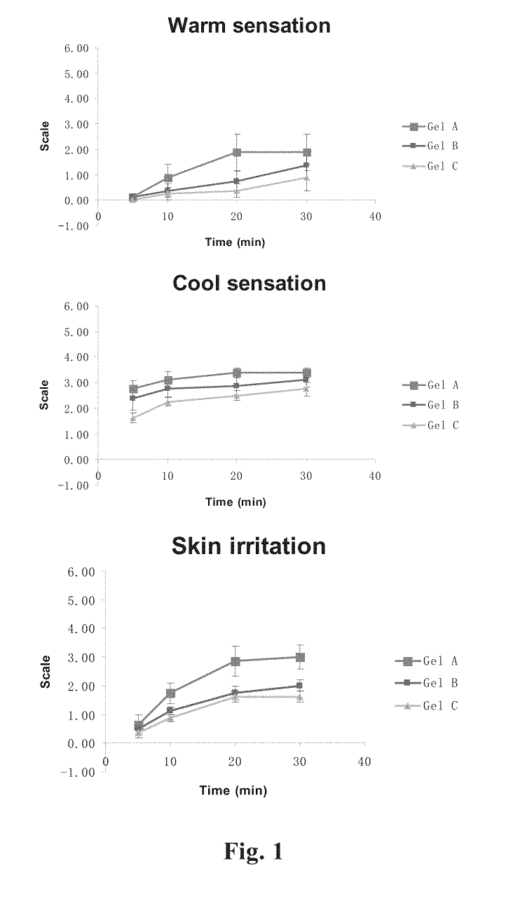

The evaluation results were shown in Table 3 below and FIG. 1.

TABLE 3
Healthy volunteer study results

| | | | | Parameter | Warming sensation | | | | | | | | | | | |
|---|---|---|---|---|---|---|---|---|---|---|---|---|---|---|---|---|
| | | | | Time | 0 = neutral; 1 = slightly warm; 2 = warm; 3 = hot; 4 = very hot | | | | | | | | | | | |
| | | Gender | | (min) | 5 | | | 10 | | | 20 | | | 30 | | |
| Volunteer # | Age | Male | Female | Gel | A | B | C | A | B | C | A | B | C | A | B | C |
| 1 | 31 | 1 | 0 | — | 0 | 0 | 0 | 0 | 0 | 0 | 0 | 0 | 0 | 0 | 0 | 0 |
| 2 | 41 | 1 | 0 | | 0 | 0 | 0 | 0 | 0 | 0 | 0 | 0 | 0 | 0 | 0 | 0 |
| 3 | 41 | 0 | 1 | | 0 | 0 | 0 | 0 | 0 | 0 | 0 | 0 | 0 | 0 | 0 | 0 |
| 4 | 64 | 1 | 0 | | 0 | 1 | 0 | 0 | 0 | 0 | 0 | 0 | 0 | 0 | 0 | 0 |
| 5 | 37 | 0 | 1 | | 0 | 0 | 0 | 0 | 0 | 0 | 4 | 2 | 0 | 4 | 3 | 0 |
| 6 | 27 | 1 | 0 | | 0 | 0 | 0 | 2 | 2 | 2 | 3 | 2 | 2 | 3 | 3 | 2 |
| 7 | 27 | 0 | 1 | | 0 | 0 | 0 | 4 | 0 | 0 | 4 | 0 | 0 | 4 | 4 | 4 |
| 8 | 31 | 1 | 0 | | 1 | 0 | 0 | 1 | 1 | 0 | 4 | 2 | 1 | 4 | 1 | 1 |
| Total | — | 5 | 3 | Total | 1 | 1 | 0 | 7 | 3 | 2 | 15 | 6 | 3 | 15 | 11 | 7 |
| Average | 37.38 | — | — | Average | 0.13 | 0.13 | 0 | 0.88 | 0.38 | 0.25 | 1.88 | 0.75 | 0.38 | 1.88 | 1.38 | 0.88 |
| SD | 12.14 | — | — | SD | 0.35 | 0.35 | 0.00 | 1.46 | 0.74 | 0.71 | 2.03 | 1.04 | 0.74 | 2.03 | 1.69 | 1.46 |
| % | — | 62.5 | 37.5 | SEM | 0.13 | 0.13 | 0.00 | 0.52 | 0.26 | 0.25 | 0.72 | 0.37 | 0.26 | 0.72 | 0.60 | 0.52 |
| | | | | Parameter | Cooling sensation | | | | | | | | | | | |
| | | | | Time | 0 = neutral; 1 = slightly cool; 2 = cool; 3 = cold; 4 = very cold | | | | | | | | | | | |
| | | Gender | | (min) | 5 | | | 10 | | | 20 | | | 30 | | |
| Volunteer # | Age | Male | Female | Gel | A | B | C | A | B | C | A | B | C | A | B | C |
| 1 | 31 | 1 | 0 | — | 1 | 1 | 1 | 2 | 2 | 2 | 3 | 2 | 2 | 3 | 3 | 3 |
| 2 | 41 | 1 | 0 | | 2 | 2 | 2 | 2 | 2 | 2 | 3 | 2 | 2 | 3 | 2 | 2 |
| 3 | 41 | 0 | 1 | | 3 | 1 | 1 | 3 | 2 | 3 | 3 | 2 | 3 | 3 | 2 | 3 |
| 4 | 64 | 1 | 0 | | 3 | 1 | 2 | 3 | 2 | 2 | 3 | 2 | 3 | 3 | 3 | 3 |
| 5 | 37 | 0 | 1 | | 3 | 3 | 2 | 4 | 3 | 2 | 4 | 4 | 2 | 4 | 4 | 2 |
| 6 | 27 | 1 | 0 | | 3 | 4 | 2 | 3 | 3 | 2 | 3 | 3 | 2 | 3 | 3 | 2 |
| 7 | 27 | 0 | 1 | | 3 | 4 | 1 | 4 | 4 | 2 | 4 | 4 | 3 | 4 | 4 | 4 |
| 8 | 31 | 1 | 0 | | 4 | 3 | 2 | 4 | 4 | 3 | 4 | 4 | 3 | 4 | 4 | 3 |
| Total | — | 5 | 3 | Total | 22 | 19 | 13 | 25 | 22 | 18 | 27 | 23 | 20 | 27 | 25 | 22 |
| Average | 37.38 | — | — | Average | 2.75 | 2.38 | 1.63 | 3.13 | 2.75 | 2.25 | 3.38 | 2.88 | 2.50 | 3.38 | 3.13 | 2.75 |
| SD | 12.14 | — | — | SD | 0.89 | 1.30 | 0.52 | 0.83 | 0.89 | 0.46 | 0.52 | 0.99 | 0.53 | 0.52 | 0.83 | 0.71 |
| % | — | 62.5 | 37.5 | SEM | 0.31 | 0.46 | 0.18 | 0.30 | 0.31 | 0.16 | 0.18 | 0.35 | 0.19 | 0.18 | 0.30 | 0.25 |

| | | | | | Skin irritation | | | | | | | | | | | | |
|---|---|---|---|---|---|---|---|---|---|---|---|---|---|---|---|---|---|
| | | | | | 0 = normal skin; 1 = mild erythema; 2 = mild erythema w/ mild pain; 3 = mild | | | | | | | | | | | | |
| | | | | Parameter | erythema w/ moderate pain; 4 = moderate erythema w/ moderate pain; 5 = | | | | | | | | | | | | |
| | | | | Time | severe erythema w/ severe pain; 6 = severe erythema w/ blister | | | | | | | | | | | | |
| | | Gender | | (min) | 5 | | | 10 | | | 20 | | | 30 | | | Withdrawal |
| Volunteer # | Age | Male | Female | Gel | A | B | C | A | B | C | A | B | C | A | B | C | time (min) |
| 1 | 31 | 1 | 0 | — | 0 | 1 | 1 | 1 | 1 | 1 | 1 | 1 | 1 | 2 | 2 | 1 | |
| 2 | 41 | 1 | 0 | | 1 | 1 | 1 | 2 | 2 | 1 | 2 | 2 | 2 | 2 | 2 | 2 | |
| 3 | 41 | 0 | 1 | | 0 | 0 | 0 | 1 | 1 | 1 | 1 | 1 | 2 | 2 | 1 | 2 | |
| 4 | 64 | 1 | 0 | | 0 | 0 | 0 | 1 | 1 | 0 | 3 | 1 | 2 | 2 | 2 | 2 | |
| 5 | 37 | 0 | 1 | | 1 | 1 | 0 | 1 | 1 | 1 | 4 | 2 | 1 | 4 | 2 | 1 | 22 |
| 6 | 27 | 1 | 0 | | 0 | 0 | 0 | 2 | 1 | 1 | 3 | 2 | 2 | 3 | 2 | 2 | 25 |
| 7 | 27 | 0 | 1 | | 3 | 0 | 0 | 4 | 1 | 1 | 5 | 3 | 2 | 5 | 3 | 2 | 24 |
| 8 | 31 | 1 | 0 | | 0 | 1 | 1 | 2 | 1 | 1 | 4 | 2 | 1 | 4 | 2 | 1 | 25 |
| Total | — | 5 | 3 | Total | 5 | 4 | 3 | 14 | 9 | 7 | 23 | 14 | 13 | 24 | 16 | 13 | |
| Average | 37.38 | — | — | Average | 0.63 | 0.50 | 0.38 | 1.75 | 1.13 | 0.88 | 2.88 | 1.75 | 1.63 | 3.00 | 2.00 | 1.63 | |
| SD | 12.14 | — | — | SD | 1.06 | 0.53 | 0.52 | 1.04 | 0.35 | 0.35 | 1.46 | 0.71 | 0.52 | 1.20 | 0.53 | 0.52 | |
| % | — | 62.5 | 37.5 | SEM | 0.38 | 0.19 | 0.18 | 0.37 | 0.13 | 0.13 | 0.52 | 0.25 | 0.18 | 0.42 | 0.19 | 0.18 | |

It was found that a combination of 3.6 wt % peppermint oil and 1.4 wt % menthol offered the maximum “cooling effect”.

When a hydrogel prototype comprising methyl salicylate starts to show mild irritation in 1 of 8 healthy subjects, the concentration of the methyl salicylate comprised in the hydrogel prototype is considered the highest concentration that can be tried safely in patients with hypertrophic scars. Methyl salicylate showed irritation at 11 wt % based on the total weight of the hydrogel composition. Based on the results, the hydrogel composition of Example 2 which contains 3.6% peppermint oil, 1.4% menthol and 2.5% methyl salicylate was a good-efficiency hydrogel composition in terms of cooling sensation, the warming sensation and irritation/erythema,

Examples 1 and 3 elicited a weaker cooling sensation when compared to Example 2 suggesting certain amount of methyl salicylate is required for offering a stronger cooling effect. Example 4 has an even lower cooling effect. Example 5 elicited similar sensations when compared to Example 2 but slightly more irritating. Example 7 offered similar effects but has an increased oily feeling. However, the hydrogel compositions of Examples 1-7 all exhibited acceptable cooling sensation, warming sensation and irritation/erythema, and are good materials for closed wound application.

2. Testing in Patients with Hypertrophic Scars

2.1 Exploratory Study

In an exploratory study, the effects of hydrogel compositions of Examples 2 (i.e., CQ-01) (with a gauze on which the respective hydrogel composition was disposed) were evaluated in 2 well-established patients with intractable pruritus associated with burn-induced hypertrophic scars (JW score: 90-100). The active arm (CQ-01) had the hydrogel applied at around 0.2 g/cm² in the areas of highest itchiness according to the patients for 8 h. Both patients were not able to have good quality sleep for around 2 years and both had severe depression and one with suicidal ideation.

It was found that both patients reported quick symptomatic relief to a tolerable range and were able to sleep un-interrupted over-night on the day of the application. Importantly, there was no adverse reaction noted with the treatment. The patients reported very positive responses to these hydrogel compositions subjectively.

2.2 Prospective Controlled Study

Prompted by this encouraging positive clinical response, a prospective, double-blinded, multicenter, controlled study was planned to evaluate the efficacy and safety of CQ-01. Burn units from three major hospitals in China, including (a) Institute of Burn Research, Southwest Hospital, Third Military Medical University, Chongqing (with 220 beds for burn patients); (b) the Zhengzhou First People's Hospital, Zhengzhou (with 170 beds for burn patients); and (c) Lanzhou General Hospital, Lanzhou Command of Chinese People's Liberation Army, Lanzhou (with 50 beds for burn patients), participated in this clinical study. All principal investigators of each study site have been involved in the care of burn patients for decades. IRB approval was obtained for all 3 units to conduct the trial under a protocol and a written informed consent form,

The symptoms were assessed using a modified visual analog scale (0-100) of scar pruritus (JW Scale; see Table 4). In the experience, this expanded scale is a sensitive tool for patients to indicate symptoms improvement or deterioration compared to other described smaller scales within ranging from 0-5^{[10, 11]} or 0-10^{[12]}. There is also a good correlation between the descriptor of the symptoms and the pattern of sleep disturbance. For those patients with discrepancies, the higher level of the score was reported. The patients were taught the use of JW scale and the full assessment was applied before treatment as baseline. A visual analog scale assessment was adopted subsequently for all time points based on the descriptors.

TABLE 4
The JW scale for the assessment of pruritus in burn patients with
hypertrophic scars

| | | | Sleep |
|---|---|---|---|
| | | | Affected^{b-d} |
| | | | (only for |
| JW | | | Initial |
| Scale | Pruritus^{a} | Descriptor | assessment) |
| 0 | None | | No |
| 1-9 | Mild | Occasional scratching | No |
| 10-19 | (0-2 spells/5 min) | Occasional scratching with occasional distraction of focus | No |
| 20-29 | Moderate | Scratch regularly and mild distraction from regular activities | No |
| 30-39 | (3-6 spells/5 min) | Scratch regularly with definitive distraction from regular activities | No |
| 40-49 | Severe | Frequent scratching, mild distraction | Yes, + |
| 50-59 | (6-20 spells/5 min) | Frequent scratching, definitive distraction | Yes, + |
| 60-69 | | Frequent scratching, almost unbearable | Yes, ++ |
| 70-79 | Intractable | Constant scratching, unbearable, mild depression | Yes, ++ |
| 80-89 | (>20 spells/5 min) | Constant scratching, moderate depression | Yes, +++ |
| 90-100 | | Constant scratching, suicidal tendency | Yes, +++ |

^{a}Spell = each of pruritus episode lasting up to 5 seconds
^{b}Sleep affected = + (1-3 awakenings per night due to pruritus): ++ (4-10 awakenings), +++ (≧10 awakenings)
^{c}The sleep disturbance usually parallels that of the pruritic symptoms but for the small number of patients with discrepancies in these two categories, we recorded the higher score for the initial assessment. As patients were treated for different areas with different treatment, only the visual analog approach for the descriptor was used with the subsequent assessments.
^{d}For the assessment in this study, we adopted a comprehensive assessment on the initial assessment and then used the visual analog approach for the subsequent assessments at different time points.

To ensure that the study was adequately powered statistically, assuming that a clinical significant improvement in the score was 20, the projected improvements are 34 and 7 with treatment arm and control arm, respectively (based on investigators' projections), and assuming a split alpha of 0.025 (assuming that an interim analysis may be conducted) and a power (1−βa) of 0.80, it was estimated that 66 patients would be required for this clinical study based on the following formula:^{[13]}

$$N = \frac{{( {Z}_{α} + {Z}_{β} )}^{2} \times ( {σ}_{1}^{2} + {σ}_{2}^{2} )}{{( {μ}_{1} - {μ}_{2} - δ )}^{2}}$$

wherein, Z_{α}=1.96, Z_{β}=0.84, μ1=34, μ2=7, σ1=19, σ2=7, δ=20.

Z_{α} and Z_{β} are Z values when alpha is 0.025 and 1−β is 0.8, respectively. Delta is the score improvement as stated above. σ₁ and σ₂ are projected improvements with treatment arm and control arm, respectively. σ₁ and σ₂ are corresponding standard deviations with treatment arm and control arm, respectively.

To ensure that the study was sufficiently powered, we intentionally added 8 more patients (around 10%) to the study size and therefore a total of 74 patients were planned in the prospective study to demonstrate the efficacy and safety of CQ-01. The study patients recruited into this study were well characterized and all had JW scores of ≧40 (i.e., all with sleep disturbance). Sixty-eight of the 74 patients (91.9%) were inpatients and 6 were outpatients. Inclusion criteria included (a) age between 16-65, (b) hypertrophic scar formed within one year of burn and had a JW scores of ≧40, and (c) have three different burn areas that can be used for testing. Exclusion criteria included (a) history of skin allergies, eczema, bacterial, or fungal infection of the skin, (b) history of mental illness not related to the scars, (c) female patients who were pregnant or were breast feeding their child, (d) history of cancer, diabetics, chronic liver, or kidney diseases, human immunodeficiency virus infection, and other chronic illnesses, (e) patients who cannot understand the JW scale and the visual analog assessment according to the physician's assessment, and (t) patients who are unlikely going to be compliant with the assessments conducted during the study period. Written informed consent was obtained from all patients in this study.

The investigator assigned three roughly equal size areas with pruritic hypertrophic scars and randomized these areas to be treated with three different applications, respectively:

(a) CQ-01 (active arm) and covered with plain gauze on top;

(b) guar-gum hydrogel without any active ingredients (gel control) (composition: water: 82.40%, cremophor EL: 2.0%, HPG: 5.2%, CMG: 0.55%, methyl paraben: 0.22%, propyl paraben: 0.11%, boric acid: 0.47%, zinc sulphate heptahydrate: 0.05%, zinc oxide: 0.15%, glycerol: 5.6%, borax: 1.3%, monosodium phosphate dehydrate: 1.95%) and then covered with plain gauze on top; and

(c) plain gauze cover (negative or gauze control).

Each of the treatment areas was labeled as A, B, or C. The active arm (CQ-01) and the gel control arm had the gel applied at around 0.2 g/cm² with an average tested area of >50 cm², respectively (Table 5). The treatment lasted for 24 hours. If the patients experienced any irritation, the treatment was discontinued, and if the patients agreed to provide the scores, the assessment would continue for the entire observation period (7 days post-treatment). Investigators who were not involved in the treatment application/assignment assessed the response of patients using the JW scale and the visual analog approach according to Table 1, During the 24-hour treatment, the patients were asked to assess the pruritus intensity based on the visual analog scale at 0.5, 1, 3, 6, 12, and 24 hours after the treatment application. The same assessment was also made at the end of Day 2, 3, 4, 5, 6, 7, and 8 after the end of treatment on Day 1 with all gels and gauzes were removed. All potential adverse events were recorded. Patients were blinded to the treatment at each site as these were all covered by gauze. However, it is admitted that it was impossible to totally blind the patients due to the physical presence of the medical gels (for both CQ-01 and the gel control), as well as the scent and sensation generated by the additives of CQ-01. Therefore, strictly speaking, this was not a double-blinded study even though the investigators tried their best to control the variables.

TABLE 5
Demographics, clinical profiles and the study treatment area
characteristics of the patients in the prospective controlled study

| Characteristics | Data |
|---|---|
| Total number of patients | 74 |
| Male | 65 (88%) |
| Age (years) | |
| Mean (SD) | 38 (11) |
| Types of burn | |
| Thermal | 59 (80%) |
| Electric | 7 (9%) |
| Chemical | 8 (11%) |
| Depth of burn (may have different depths in different areas) | |
| Second-degree superficial | 32 (43%) |
| Second-degree deep | 73 (99%) |
| Third-degree | 53 (72%) |
| Burn area (% TBSA) | |
| Mean (SD) | 41 (23) |
| Second-degree superficial | 12 (11) |
| Second-degree deep | 22 (14) |
| Third-degree | 20 (19) |
| Time for the burn to heal (days) | |
| Mean (SD) | 62 (51) |
| Time from healed to this clinical study (days) | |
| Mean (SD) | 226 (413) |
| Testing articles applied to: CQ-01:gel control:gauze control | |
| Head/face/neck | 5:4:2 |
| Extremities | 38:34:39 |
| Body | 31:36:33 |
| Estimation of the area applied (cm²) - Mean (SD) | |
| CQ-01 | 85 (83) |
| Gel Control | 75 (60) |
| Gauze Control | 62 (51) |

Four subjects who reported irritation consented later to further testing with a modified version of the hydrogel composition without methyl salicylate (CQ-02 sheet) for another 24 hours at around 0.2 g/cm² and covered with gauze. This was to test whether the irritation was related to methyl salicylate and whether the hydrogel with merely peppermint oil and menthol as active ingredients could alleviate the symptoms,

Results of this prospective controlled study:

As described above, a prospective controlled study was then designed and conducted to evaluate the efficacy and safety of CQ-01 in patients with severe and intractable pruritus associated with burn-induced hypertrophic scars. Three major Burn Centers in China participated in this study and the number of patients enrolled in each center was: 35 in Chongqing, 21 in Zhengzhou, and 18 in Lanzhou. A summary of demographics and clinical characteristics of the 74 patients are summarized in Table 5. There were no differences in demographics of patients, clinical profiles, and treatment characteristics among the three centers. The majority of the patients had thermal burns (n=59, 79.7%) and the rest were electric and chemical burns (n=7 and 8, respectively). All patients suffered from at least second-degree or third-degree burns. All patients had severe and intractable pruritus associated with their hypertrophic scars with interrupted sleep (JW score ≧40),

Safety:

Of the 74 patients studied, only 6 patients (8.1%) reported any adverse events. All 6 patients reported mild to severe irritation from 3 min to 2 hours after the CQ-01 application, Two of them reported to be mild and continued to complete the 24-hour of CQ-01 treatment. The other 4 reported moderate to severe irritation, and the treatment was discontinued immediately: 2 patients did not provide any input on the JW score response, and 2 (both discontinued at 2 hours) after CQ-01 application and only one of them provided treatment responses for the entire 8 days. The main description of the irritation was a burning sensation. All patients had quick relief when CQ-01 was removed (<1 hour in all cases). There was no visible skin redness (n=4) to very mild redness/erythema (n=2) in these 6 patients. There were two patients with mild irritation and received CQ-01 treatment for 24 hours; one had a tiny blister (<1 cm²; applied area=45 cm²) and the other patient had a small area of broken epidermis that was also observed in the other areas. The investigator considered these findings were more related to the patient's fragile epidermis covering the hypertrophic scars. There was no other long-term adverse effect observed in these 6 patients,

Efficacy:

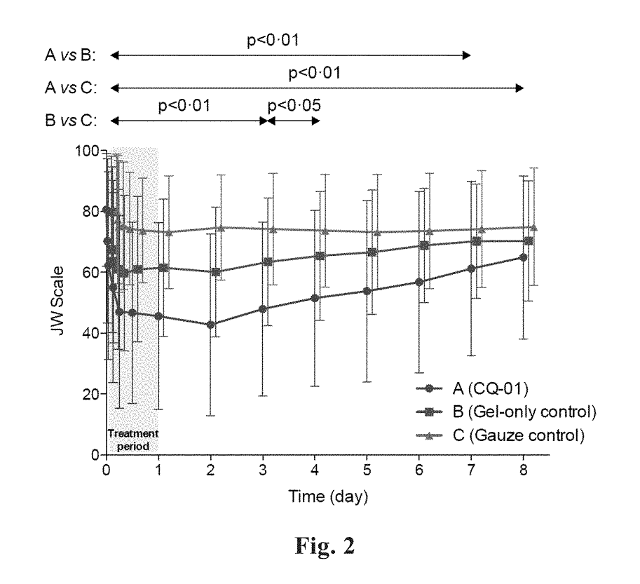

Four patients did not provide any JW scores after the application due to withdrawal. For the 70 patients who completed the JW scale assessment, 69 received a 24-hour of CQ-01 treatment while one discontinued the treatment at 2 hours because of irritation. The effects of CQ-01, gel control, and gauze negative control based on per protocol (PP) analysis are summarized in FIG. 2. There were a number of observations. First, the average baseline JW score for this group of patients was around 80±20, confirming that this group of patients represented those with severe and intractable pruritus associated with burn-induced hypertrophic scars. As all of them had a score of ≧40, they all had interrupted sleep due to pruritus. Second, the negative-control group with gauze cover only had an average JW score drop of 7 during the 24-h treatment period, highlighting the placebo effect or attention benefit with the medical care, contact effect (of the gel), or the potential spill-over effect of the treatment from other parts of the body. Third, gel control showed an additional reduction in the average JW score by 11 compared to gauze control during the 24-h treatment period. This effect occurred rapidly, within 30 minutes of application and lasted up to Day 4 (p<0.05). There was no significant difference between Day 5-8 when compared to the gauze control. Fourth, CQ-01 had a similar immediate effect on the average JW score in the first 3 hours of application. However, CQ-01 showed an additional effect beyond the gel physical effect from 3-6 hours after the application (p<0.01), The effect was more pronounced after 6 hours of application and resulted in an average reduction in the JW score of 30 as compared to gauze negative-control on Day 2 (p<0.01 compared to both gel control and gauze control). From Day 3, the effect began to diminish but still clinically significant compared to gauze negative-control with an average reduction in the JW score of 20-32 (p<0.01) until Day 5 and about 15 (p<0.01) at the end of Day 8 was observed, indicating the durability of the CQ-01 effect.

Time to Onset of Action:

The onset of clinical effect took a median of 1 hour to reduce JW score by 10 points for both CQ-01 and gel control. For a JW score of 20 and 30 points reduction, the median onset time for CQ-01 was 6 hours whereas the gel control was 18 hours (p<0.01) and 192 hours (p<0.01), respectively.

ITT Analysis for Proportion of Patients with JW Score of <40 and <60:

Based on Intention-to-treat analysis (n=74), we examined the proportion of patients that had scores below 40 during treatment (as this is the cut-off for sleep disturbance) using the visual analog assessment. The investigators knew that these patients were treated with the controls at the same time, and therefore; the visual analog assessment might be biased based on the overall feelings of the patients, Even with this limitation; 29/74 (39.2%) patients reported JW scores of <40 on Day 2 compared to 9 (12.2%) with gel control and 0 (0.0%) with gauze negative-control treatment (p<0.01) (Table 6A). The proportion of patients with JW scores of <40 was higher for the CQ-01 treatment throughout the observation period. As patients with a JW score of 40-59 have only mild sleep disturbance; we also assessed the proportion of the patients with scores below 60 before and during the treatment period (Table 6B). Note that around 12% of patients in our study proportion only had mild sleep disturbance before the study commenced. During treatment and when patients were asked for visual analog score of the individual treated area, CQ-01 was able to achieve 45/74 (60.8%) patients with a JW score of less than 60 on Day 2 compared to 25/74 (33.8%; p<0.01) with gel control and 11/74 (14.9%; p<0.01) with gauze control. The beneficial effect of CQ-01 was observed over the entire duration of treatment and observation period (Table 6),

TABLE 6
A. Number and percentage of patients with JW
score below 40 (ITT; n = 74)

| Days | CQ-01 | Gel-only control | Gauze control |
|---|---|---|---|
| 0 | 0 (0·0%) | 0 (0·0%) | 0 (0·0%) |
| 1 | 24 (32·4%) | 9 (12·2%) | 1 (1·4%) |
| 2 | 29 (39·2%) | 9 (12·2%) | 0 (0·0%) |
| 3 | 21 (28·4%) | 7 (9·5%) | 1 (1·4%) |
| 4 | 19 (25·7%) | 4 (5·4%) | 1 (1·4%) |
| 5 | 15 (20·3%) | 2 (2·7%) | 2 (2·7%) |
| 6 | 16 (21·6%) | 2 (2·7%) | 2 (2·7%) |
| 7 | 11 (14·9%) | 2 (2·7%) | 2 (2·7%) |
| 8 | 9 (12·2%) | 2 (2·7%) | 2 (2·7%) |

TABLE 6
B. Number and percentage of patients with
JW score below 60 (ITT; n = 74)

| Days | CQ-01 | Gel-only control | Gauze control |
|---|---|---|---|
| 0 | 10 (13·5%) | 10 (13·5%) | 11 (14·9%) |
| 1 | 41 (55·4%) | 29 (39·2%) | 17 (23·0%) |
| 2 | 45 (60·8%) | 25 (33·8%) | 11 (14·9%) |
| 3 | 42 (56·8%) | 21 (28·4%) | 14 (18·9%) |
| 4 | 38 (51·4%) | 23 (31·1%) | 16 (21·6%) |
| 5 | 36 (48·6%) | 26 (35·1%) | 15 (20·3%) |
| 6 | 30 (40·5%) | 21 (28·4%) | 12 (16·2%) |
| 7 | 27 (36·5%) | 16 (21·6%) | 15 (20·3%) |
| 8 | 23 (31·1%) | 14 (18·9%) | 14 (18·9%) |

Note:
Patients reported different levels of pruritus in different burn areas and therefore, there was a difference in the JW score of different treatment areas.

Extent of Response:

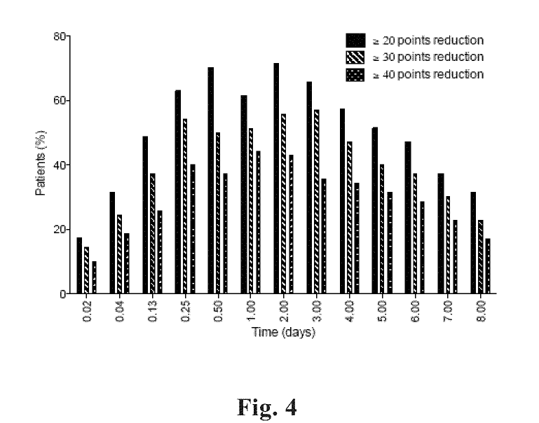

The percentage of patients with a drop of 20, 30, and 40 points in the JW score after CQ-01 application was analyzed (FIG. 4). With CQ-01, 43 patients (58.1%) had JW scores reduced by ≧20 points after 24 h treatment compared to 28 patients (37.8%; p<0.05) for gel control and 6 patients (8.1%; p<0.01) for gauze control]. Among them, 36 patients (48.6%) had scores reduced by ≧30 points indicating that about 50% of patients reached a category of a lower severity (e.g., from severe pruritus to moderate pruritus in the JW scale). On the other hand, the gel control and gauze negative control had only 18 patients (24.3%; p<0.01) and 5 patients (6.8%; p<0.01) with ≧30 points reduction, respectively. In addition, 31 patients (41.9%) had a score reduction of ≧40 compared to 13 patients (17.6%; p<0.01) with the gel control arm and 3 patients (4.1%; p<0.01) in the gauze control]. At the end of the observation period (Day 8), 12 patients (16.2%) on the CQ-01 arm still reported a reduction of JW score by ≧40 points reduction (p<0.05 compared to the two control arms).

Durability of Response:

For the duration of the JW scale reduction, CQ-01 induced a reduction in JW score of >10 for a median of 168 hours, a reduction in JW score of >20 for 119 hours, and a reduction of >30 for 93 hours.

Demographic and Clinical Associations:

There was no association between the response to CQ-01 and any of the demographic data, burn-related parameters (including the severity and the area of the burn), as well as the duration needed for the initial recovery and the duration between wound recovery and the initiation of CQ-01 therapy. CQ-01 was effective for burns caused by different etiologies.

Further Characterization of the Patients with Irritation with CQ-01:

To further clarify the mechanism for irritation developed in the small subset of patients undergoing CQ-01 application, CQ-02 sheet was applied to these patients with an aim to elucidate whether methyl salicylate was responsible for the irritation. Four patients that developed irritation after CQ-01 application agreed to participate in this follow-up study. When CQ-02 was applied, 2 patients had no irritation with CQ-02 applied for the full 24 hours and reported improvement in symptoms for more than 2 days. However, the other 2 patients still experienced irritation and removed the CQ-02 gel after 130 minutes and 6 hours of application respectively, suggesting that these patients might have irritable hypertrophic scars (as these patients also found the gel alone control to be irritating to them). Interestingly, both patients reported symptomatic improvement with CQ-02 (one patient who had CQ-02 for 130 min reported symptomatic improvement for 3 days; and the other patient who had CQ-02 for 6 hours had improvement for 1 day). The irritation disappeared in the 2 patients with irritation shortly after the removal of CQ-02. There were no other adverse events reported.

2.3 Follow-Up Studies

Finally, 3 follow-up studies were pursued to examine the following parameters for CQ-01:

(a) Reproducibility of CQ-01 Treatment (n=10)—

In this study, 10 patients were tested for one additional treatment for 24 hours at average 0.2 g/cm² and covered with gauze at least 2 weeks after the effect of the first CQ-01 treatment had worn off, the average baseline JW score was back to 79±20 (compared to 79±22 before the first application of CQ-01). In 3 patients that were still in the hospital after the second treatment, they consented to a third treatment for 24 hours at average 0.2 g/cm² and covered with gauze at least 10 weeks after the second treatment. The same assessment using JW scale for 24 h treatment followed by the same observation period (for a total of 8 days) was adopted.

(b) Guar-Gum Hydrogel Versus Silicone-Based Gel (n=20)—

As the gel control provided a beneficial clinical effect, this study was to test whether silicone oil-based gel also had any effect. The gel control; Silicone gel sheet (Nanyang Huibo Biotechnology Co., Ltd.; Wei Shi Road; Industrial Park; Nanyang, Henan Province; China) and a gauze only control arm were applied in 20 patients with pruritus associated with hypertrophic scars, respectively. The treatment protocol was to apply the treatment for 24 hours at average 0.2 g/cm² and observation period was for 5 additional days (i.e., total 6 days). Evaluation at the same time points during treatment and follow-up were performed as in the prospective controlled study.

(c) CQ-01 Versus Aqua Cream with the Same Additives (n=10)—

To test whether the guar gum gel was critical in the clinical activity of CQ-01, the effect of CQ-01 was compared to an aqua cream (available from Neutrogena company) formulated with the same concentrations of peppermint oil, menthol; and methyl salicylate in CQ-01 in 10 patients. The treatment was conducted for 24 hours at average 0.2 g/cm² with an observation period of 5 additional days (i.e., total 6 days). Evaluation at the same time points during treatment and follow-up were performed as in the prospective controlled study.

Results of these Follow-Up Studies:

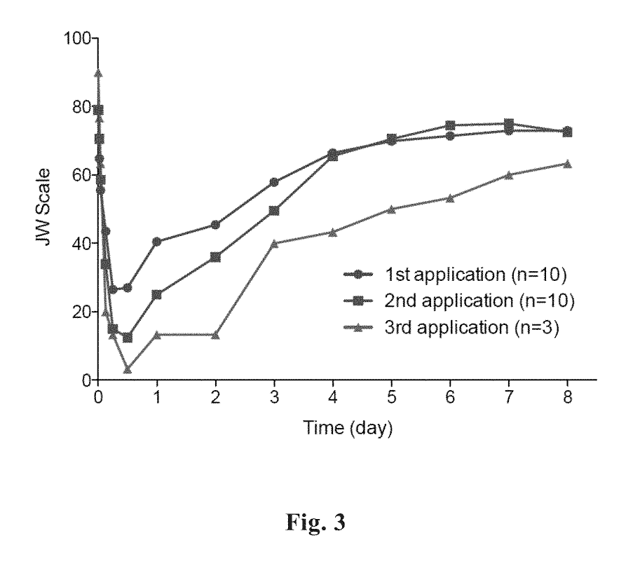

In additional follow-up studies in 10 patients, the effect of CQ-01 was assessed in a second treatment with a duration between treatments of >9 days. As depicted in FIG. 3, the second treatment of CQ-01 was as effective as the first treatment at most time points (and numerically more effective in some time points). The second treatment reduced the average JW score to below 40 from 3 hours to 2 days with a nadir average JW score of 13 in 12 hours. Three patients volunteered to stay for a third treatment in the hospital for detailed assessment (with duration between treatments >9 days). With the third CQ-01 application, the average JW score was reduced to below 40 for up to Day 3 (n=3). Although the number of patients studied was small, there was no evidence of tachyphylaxis noted with CQ-01 treatment.

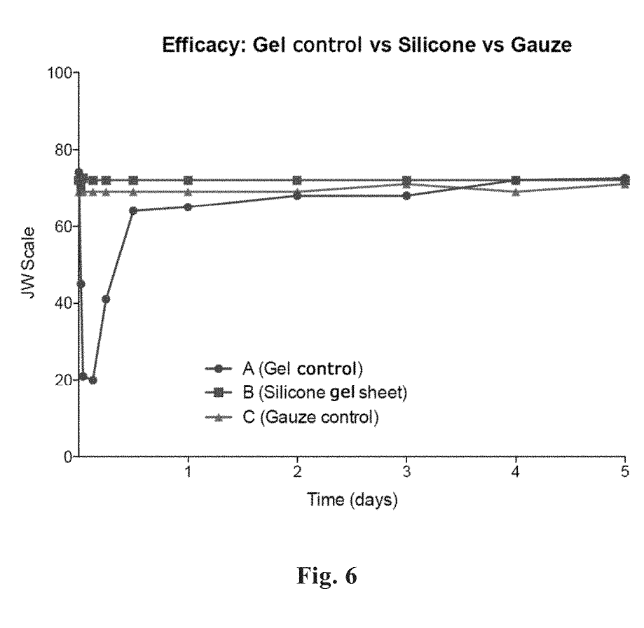

To further define the role of the guar-gum hydrogel, we conducted two additional follow-up clinical studies. First, in 20 patients, gel control treatment (i.e., without active ingredients) was compared to silicone gel sheet (oil-based) and gauze only (negative control) applications, as described above. The guar-gum hydrogel showed the expected effect on the average JW score. However, silicone gel did not induce any meaningful improvement in the JW scale compared to the gauze control (as shown in FIG. 6).

In another study of 10 patients, an aqua cream with the same concentrations of additives (peppermint oil, menthol, and methyl salicylate) was applied directly to the studied area (gauze covered), and with gauze negative control. The cream produced the same effect in reducing the average JW score quickly to around 20±29 in 30 minutes but bounced back to 50±32 in 12 hours and back to the baseline in 24 hours. There was no lasting effect seen 24 hours after the application, indicating that the use of guar gum was involved in the induction of a long lasting effect not seen with an aqua cream loaded with the same concentrations of additives in CQ-01.

### Medical Ethics Committee Approvals and Statistics

All studies were approved by the Southwest Hospital Ethics Committee (Approved No. 2013-30) and were conducted based on the ethical principles for medical research involving human subjects detailed in the Declaration of Helsinki (adopted in June 1964 and with the latest amendments adopted by 59^{th} World Medical Association General Assembly in Seoul, Republic of Korea in 2008).

The statistical considerations for the sample size for the prospective controlled study were given previously. All study results were analyzed based on intention-to-treat (ITT) analysis (n=74). There were 4 patients dropped out early from the study prior to the first VAS assessment time point without providing any scores for the assessment. Therefore, only 70 patients' dataset were available for the assessment of the scores of the JW scale and based on per protocol analysis.

### CONCLUSION

This is the largest reported prospective controlled study that evaluated any treatment modality for severe pruritus in patients with burn-related hypertrophic scars. The invention showed a number of important points. First, the hydrogel composition according to the present invention is safe and effective symptomatic treatment for severe and intractable pruritus associated with burn-related hypertrophic scars. Its reported adverse event is irritation (in terms of burning sensation) in 8.1% (CQ-01) of the patients that subsides quickly upon the composition's removal. The efficacy of the hydrogel composition is also independent of any demographic, burn-related factors and etiology. Second, the hydrogel composition is effective with repeated treatment with no evidence of tachyphylaxis. Third, the invention also revealed placebo/attention effects as well as gel-alone effective treatment effects for the pruritus symptoms. Finally, a new treatment paradigm (as shown in FIG. 5) is proposed based on the results reported in this invention.

In cases of burn and extensive skin damage, most medical attention is rightly devoted to the immediate care and recovery of patients. However, the long-term devastating complications of distress, sleep deprivation, inability to concentrate and depression caused by severe to intractable pruritus associated with burn-related hypertrophic scar remain neglected. This represents one of the major unmet medical needs in this technical field. Teaming up with specialists from medical and materials sciences, the invention set a goal to develop more effective treatment modalities for this medical challenge. As the pathogenesis for the pruritic symptoms for hypertrophic scars is unknown, the inventors identified several key facts in medical literature and coupled those with their experience to explore further. From the inventors' experiences, dry scar surfaces and the use of oil-based treatment can exacerbate the pruritic symptoms, but massaging scars can alleviate the symptoms. This may be related to the release of certain local mediators.

Based on theoretical and experimental investigations, the inventors selected a galactose-mannose polysaccharide-based medical hydrogel based on guar gum derivates. Guar gum is produced from the ground endosperm of guar beans that is widely used in food and industrial products and is biodegradable. It is found that the guar gum derivates based hydrogel, when adjusted to around pH 6 consistent with physiological skin condition, is moisture-permeable, low-irritating and non-sticky to the skin surface and can be readily removed, and when it possesses high water content (>70%), it can help moisturize and soften scar tissues. The large heat capacity also provides an antipruritic effect by buffering the skin surface temperature. This hydrogel can be used as an occlusive dressing suitable for uneven contours of hypertrophic scars. It can also serve as a drug delivery system for direct treatment of scars. Based on the information from the literature, the invention tested three ingredients: peppermint oil, menthol, and methyl salicylate to provide the desirable cooling and anti-inflammatory effect. These ingredients can also serve as preservatives and fragrance for the medical hydrogel. As these ingredients are oily, the invention adopted a proprietary amphiphilic nanoparticle platform and impregnated this with the oily ingredients. When mixed with the hydrocolloidal gel, the nanoparticle maintained the hydrophilic nature of the medical hydrogel. A range of these components were tested in healthy volunteers with the objective of identifying the prototype with the maximum cooling effect and with the minimal irritation. CQ-01 was thus developed. Two patients with severe pruritic hypertrophic scars tested in an exploratory study were so relieved symptomatically that it was convinced that a prospective study was warranted to demonstrate the efficacy and safety of CQ-01 in a systematic way.

To measure the efficacy endpoints, i.e., improvement in pruritus compared to baseline, the invention employed the JW scale, which was modified from a 10-point visual analog scale (VAS)^{[13]}. In order to capture the subtle changes experienced by patients from a day-to-day basis, the invention expanded the scale to 0-100 to enhance the sensitivity of the instrument. It is important to note that even though the JW score was established to access the severity of the symptoms with sleep disturbance as part of the assessment, the subsequent assessment during treatment with all three options treated in the same patient precluded the inventors from assessing sleep disturbance as part of the JW scale and only visual analog based on the descriptor for each area was assessed. It is obvious that there were variations with JW score in different scar areas within the same patient, but as the study size is relatively large, the investigator has made an effort in selecting regions/areas with similar JW score were reasonably similar but may not be identical.

The inventors powered the study so that a 20% difference would be shown to be statistically significant with 80% confidence assuming a one-sided alpha of 0.025. The inventors added 8 more patients (around 10%) to the study size to ensure adequate power in case of unexpected dropout (the protocol did not allowed for replacement), Both gauze control and gel control were used so that the placebo effect and also the gel effect could be assessed. The inventors tried to conduct this study in a double-blinded fashion. However, the presence of a hydrogel and the aroma and cool/warm effect of the components in hydrogel compositions made it impossible to completely blind the patients from these effects. The inventors did cover all these areas with gauze and assessed the symptoms using the JW scale according to simple A, B, and C labels.

The results were very informative and provided insights into the management of pruritus for these patients. The small improvement (of an average of 7 points in the JW scale) with gauze negative control was expected. This effect could be related to the medical attention, the placebo effect, or the “spillover” effect from treatment of other areas of the same patient. The gel control effect was unexpectedly large. The effect was immediate and fairly significant, and lasted for a few days after its removal. It is attributed to the cooling effect, the hydration, and occlusive nature of the hydrogel. What is surprising is the duration of the effect being persistent for a few days after the hydrogel was removed. This may be related to the lasting effect of the skin hydration, or a change in the pathogenetic process induced by the hydrogel application for 24 hours.

The observation that greater improvement in JW scores at CQ-01 sites compared to gel control sites after 3 hours of application suggested that the components were exerting their effect through mediators. It is interesting to note that on Day 2 after the application of CQ-01 (the best response observed), 41.4% of the patients had average JW score of less than 40 (with no projected disturbance of sleep) and 65.7% had average JW score of less than 60 (i.e., with an additional 24.3% having projected mild disturbance to sleep), which indicated that more than half of these patients would have improved quality of sleep with less than 4 awakenings per night due to pruritus. A quick immediate self-criticism in interpretation is the relative contribution of the placebo effect and gel effect that were partly addressed in Table 4. In our experience, a better sleep quality is one of the keys factors driving the quality of life in these patients. The lasting effect of CQ-01 for a few days after 24 hours of treatment also implied the probable impact of CQ-01 on the pathogenetic process of pruritus in these patients. As CQ-01 imparted a much stronger effect and for a longer time compared to either the hydrogel alone (much weaker effect) or the aqua cream with the same ingredients (much shorter duration), it was apparent that CQ-01 offered synergistic effect of this combination on the overall clinical efficacy, What was even more interesting was that when CQ-01 was applied repeatedly after a washout period, there was a trend that repeated treatment was at least the same if not more effective. This is contrary to original hypothesis that tachyphylaxis might occur. This suggests that with CQ-01 treatment, even though the pruritic symptoms may recur after treatment, the threshold for pruritus may have been reset so that additional treatment may be effective. This will need to be confirmed in a larger study and whether long-term use of CQ-01 can be spaced out to a “treat as needed” basis due to a reset of the pruritus threshold remains to be determined.

The results also showed that oil-based silicone gel was not as effective as guar-gum hydrogel. This is consistent with inventors' previous experience that oil-based gels or creams are uncomfortable to these patients. The short duration effect of the aqua cream containing peppermint oil, menthol and methyl salicylate was consistent with CQ-01 but CQ-01 produced a stronger and more durable clinical effect,

It is important to note that CQ-01 is a very safe treatment approach. There were 6 patients out of 74 (8.1%) who had mild to severe irritation in terms of burning sensation after CQ-01 application and upon removal of CQ-01, all symptoms quickly subsided and there were no other short- or long-term adverse events either locally or systemically. The fact that these patients described some pruritic symptomatic relief even with a short duration of treatment with CQ-01 was encouraging. Given the safety profile, the invention can recommend that all such patients can be treated with CQ-01 and be instructed to remove the hydrogel composition if there are any intolerable irritation symptoms.

Four of the six patients with irritation participated for further study on the underlying mechanism. The additional experiment showed that 2 of the 4 patients had no irritation after applying CQ-02 (CQ-01 without methyl salicylate), which suggested that methyl salicylate may be responsible for the irritation in some patients. Whether this is related to its physicochemical properties or to the prostaglandin metabolism is unknown. The fact that the irritation is observed within 5-30 minutes suggested that this is more likely related to the physicochemical properties of the compound. Interestingly, another 2 of the 4 patients that received CQ-02 also had irritation 130 minutes and 6 hours after the application. These data highlighted the heterogeneous nature of the pathogenetic mechanisms involved in the hypertrophic scars. The data also suggested that there was a group of patients with hypertrophic scars that were very sensitive to any stimulation. The observation that CQ-02 helped all 4 patients with CQ-01 irritation suggested that CQ-02 might have a clinical role in patients that were sensitive to CQ-01. Therefore, a treatment algorithm for patients with severe or intractable pruritus associated with burn-induced hypertrophic scar was formulated as shown in FIG. 5.

In summary, this invention represents another important step in the continuous effort to develop an effective treatment for this unmet medical need.

It would be appreciated by those skilled in the art that various changes and modifications may be made to the described exemplary embodiments without departing from the spirit and essential of the invention. Those changes and modifications fall into the scope of the appended claims.

### REFERENCES

1 Friedstat J S, Hultman C S. Hypertrophic Burn Scar Management: What Does the Evidence Show? A Systematic Review of Randomized Controlled Trials. Ann Plast Surg 2014; 72: S198 S201.
2 Nedelec B, Rachelska G, Parnell L K S, LaSalle L. Double-Blind, Randomized, Pilot Study Assessing the Resolution of Postburn Pruritus, J Burn Care Res 2012; 33: 398-406,
3 Zachariah J R, Rao A L, Prabha R, Gupta A K, Paul M K, Lamba S. Post burn pruritus—A review of current treatment options. Burns 2012; 38: 621-9.
4 Goutos I, Clarke M, Upson C, Richardson P, Ghosh S. Review of therapeutic agents for burns pruritus and protocols for management in adult and paediatric patients using the GRADE classification. Indian J Plast Surg 2010; 43: S51-S62.
5 Goutos I, Dziewulski P, Richardson P M. Pruritus in Burns: Review Article. J Burn Care Res 2009; 30: 221-8.
6 Bell P L, Gabriel V. Evidence Based Review for the Treatment of Post-burn Pruritus. J Burn Care Res 2009; 30: 55-61.
7 Phillips T J, Gerstein A D, Lordan V. A Randomized Controlled Trial of Hydrocolloid Dressing in the Treatment of Hypertrophic Scars and Keloids. Dermatol Surg 1996; 22: 775-8.
8 Eishi K, Bae S J, Ogawa F, Hamasaki Y, Shimizu K, Katayama I. Silicone gel sheets relieve pain and pruritus with clinical improvement of keloid: possible target of mast cells. J Dermatolog Treat 2003; 14: 248-52,
9 Berman B, Flores F. Comparison of a Silicone Gel-Filled Cushion and Silicon Gel Sheeting for the Treatment of Hypertrophic or Keloid Scars. Dermatol Surg 1999; 25: 484-6.
10 Morris V, Murphy L M, Rosenberg M, Rosenberg L, Holzer C E III, Meyer W J III. Itch Assessment Scale for the Pediatric Burn Survivor, J Burn Care Res 2012; 33: 419-24.
11 Matheson J D, Clayton J, Muller M J. The Reduction of Itch During Burn Wound Healing. J Burn Care Rehabil 2001; 22: 76-81.
12 Field T, Peck M, Hernandez-Reif M, Krugman 5, Burman I, Ozment-Schenck L. Postburn Itching, Pain, and Physchological Symptoms Are Reduced With Massage Therapy. J Burn Care Rehabil 2000; 21: 189-93.
13 Wu X. Clinical Study Sample Size Estimation (in Chinese). People's Medical Publishing House Co. Ltd. 2008; ISBN: 978-7-117-09985-1/R.9986.

What is claimed is:

1. A hydrogel composition comprising:
a guar gum derivative which is crosslinked;
a mint-based component;
water; and
optionally, an amphiphilic core-shell nanoparticle having a hydrophobic core and a hydrophilic shell;
wherein the amount of the mint-based component is in the range of 1-6 wt % and the amount of the water is in the range of 65-95 wt %, based on the total weight of the hydrogel composition.

2. The hydrogel composition according to claim 1, wherein the amount of the guar gum derivative is in the range of 1-7 wt % based on the total weight of the hydrogel composition.

3. The hydrogel composition according to claim 1, wherein the guar gum derivative is selected from the group consisting of hydroxypropyl guar gum, carboxymethyl guar gum, O-carboxymethyl-O-hydroxypropyl guar gum and O-2-hydroxy-3-(trimethylammonio)propyl guar.

4. The hydrogel composition according to claim 3, wherein the guar gum derivative is a combination of hydroxypropyl guar gum and carboxymethyl guar gum, and the amount of the hydroxypropyl guar gum is in the range of 1-6 wt % and the amount of the carboxymethylguar gum is in the range of more than 0 wt % to 1 wt %, based on the total weight of the hydrogel composition.

5. The hydrogel composition according to claim 1, wherein the guar gum derivative iscrosslinked by borate, glutaraldehyde and/or glyoxal.

6. The hydrogel composition according to claim 1, wherein the guar gum derivative is at least partially further crosslinked with the shell of the nanoparticle.

7. The hydrogel composition according to claim 1, wherein the amount of the nanoparticle is in the range of 0.5-2 wt % based on the total weight of the hydrogel composition.

8. The hydrogel composition according to claim 1, wherein the nanoparticle is selected from the group consisting of poly(methyl methacrylate)-chitosan nanoparticle, poly(methyl methacrylate)-polyethylenimine nanoparticle and poly(methyl methacrylate)-poly(allylamine) nanoparticle.

9. The hydrogel composition according to claim 8, wherein the nanoparticle is poly(methyl methacrylate)-chitosan nanoparticle, and wherein the poly(methyl methacrylate) constitutes the core of the nanoparticle and the chitosan constitutes the shell of the nanoparticle.

10. The hydrogel composition according to claim 9, wherein the weight ratio of the poly(methyl methacrylate) to the chitosan is in the range of 1: (3-5).

11. The hydrogel composition according to claim 1, wherein the number average particle size of the nanoparticle is in the range of 100-150 nm.

12. The hydrogel composition according to claim 1, wherein the mint-based component is encapsulated by the nanoparticle.

13. The hydrogel composition according to claim 1, wherein the mint-based component is selected from the group consisting of peppermint oil, menthol, cornmint oil and spearmint oil.

14. The hydrogel composition according to claim 13, wherein the mint-based component is a combination of peppermint oil and additional menthol, and wherein the amount of the peppermint oil is in the range of more than 0 wt % to 4 wt % and the amount of the additional menthol is in the range of 0.5-2 wt %, based on the total weight of the hydrogel composition.

15. The hydrogel composition according to claim 1, wherein the hydrogel composition does not comprise methyl salicylate.

16. The hydrogel composition according to claim 1, wherein the hydrogel composition further comprises up to 2.5 wt % methyl salicylate based on the total weight of the hydrogel composition.

17. The hydrogel composition according to claim 16, wherein the hydrogel composition comprises 1-2.5 wt % of methyl salicylate based on the total weight of the hydrogel composition.

18. The hydrogel composition according to claim 1, wherein the hydrogel composition further comprises a plasticizer.

19. A method for preparing the hydrogel composition according to claim 1, comprising the steps of:
1) providing an emulsion containing at least a part of the mint-based component; and
2) combining the emulsion containing at least a part of the mint-based component with the guar gum derivative, the remaining mint-based component if any, and a crosslinker to induce crosslinking of the guar gum derivative, thereby forming the hydrogel composition.

20. A method for preparing the hydrogel composition according to claim 19, wherein the step 1) comprises the following steps:
i) providing an aqueous emulsion of the amphiphilic core-shell nanoparticle having a hydrophobic core and a hydrophilic shell; and
ii) mixing the aqueous emulsion of the nanoparticle with at least a part of the mint-based component to form the emulsion containing at least a part of the mint-based component.

21. The method according to claim 20, wherein the concentration of the aqueous emulsion of the nanoparticle provided in step i) is in the range of 0.5-5% (w/w).

22. The method according to claim 20, wherein in step ii) the aqueous emulsion of the nanoparticle and the at least a part of the mint-based component are mixed such that the mint-based component is encapsulated by the nanoparticle.

23. The method according to claim 19, wherein the amount of the mint-based component added in step 1) is in the range of 20-100 wt % based on the total amount of the mint-based component.

24. The method according to claim 23, wherein the amount of the mint-based component added in step 1) is in the range of 20-30 wt % based on the total amount of the mint-based component.

25. The method according to claim 24, wherein the mint-based component added in step 1) is menthol, and the remaining mint-based component added in step 2) is peppermint oil.

26. The method according to claim 19, wherein step 1) further comprises adding methyl salicylate.

27. The method according to claim 19, wherein step 2) further comprises adding a plasticizer.

28. The hydrogel composition according to claim 1 for use in relieving symptoms induced by hypertrophic scars.

29. The hydrogel composition for use according to claim 28, wherein the dosage of the hydrogel composition is in the range of 0.2-0.8 g/cm² skin.

30. The hydrogel composition for use according to claim 28, wherein the hydrogel composition is in the form of dressings.

31. A method of relieving symptoms induced by hypertrophic scars, comprising: administrating to a subject in need of relieving the symptoms a pharmaceutically effective amount of the hydrogel composition according to claim 1.

32. The method according to claim 31, wherein the hydrogel composition is administered at a dosage in the range of 0.2-0.8 g/cm² skin.

33. The method according to claim 31, wherein the hydrogel composition is topically administered at a dosage in the range of 0.2-0.8 g/cm² skin and renewed every 4-6 hours.

34. A medical device comprising:
a package containing the hydrogel composition according to claim 1, and
an instruction for administration of the hydrogel composition for relieving the symptoms induced by hypertrophic scars.

35. The medical device according to claim 34, wherein the package further comprises a substrate to which the hydrogel composition is disposed, or an applicator for application of the hydrogel composition.
